(19) United States
(12) Patent Application Publication
(10) Pub. No.: US 2022/0226472 A1
(43) Pub. Date: Jul. 21, 2022

(54) Antimicrobial Quantum Dots and Methods of Tuning the Same

(71) Applicant: The Regents of the University of Colorado, a Body Corporate, Denver, CO (US)

(72) Inventor: Prashant NAGPAL, Lafayette, CO (US)

(21) Appl. No.: 17/616,491

(22) PCT Filed: Jun. 11, 2020
(86) PCT No.: PCT/US2020/037182

§ 371 Date: Dec. 3, 2021

Related U.S. Application Data
(60) Provisional application No. 62/860,124, filed on Jun. 11, 2019.
(60) Provisional application No. 62/926,204, filed on Oct. 25, 2019.

(51) Int. Cl. A61K 41/00; C09K 11/70; C09K 11/88; C09K 11/02; C09K 11/56; A61K 33/42; A61K 33/30; A61P 31/04; A61N 5/06

(52) U.S. Cl. CPC A61K 41/0057; C09K 11/70; C09K 11/88; C09K 11/02; C09K 11/565; A61N 2005/0663; A61K 33/42; A61K 33/30; A61P 31/04; A61N 5/062; A61N 5/0624; C09K 11/883

(57) ABSTRACT

The present disclosure relates in one aspect to compositions containing indium phosphide (InP) or ternary zinc cadmium telluride (Zn₁₋ₓCdₓTe) quantum dots. These quantum dots are useful as selective antimicrobial agents. Compositions containing the quantum dots described herein are nontoxic to human cells.

### CROSS-REFERENCE TO RELATED APPLICATIONS

This application claims priority under 35 U.S.C. § 119(e) to U.S. Provisional Patent Application No. 62/860,124 entitled “ANTIMICROBIAL QUANTUM DOTS AND METHODS OF TUNING THE SAME,” filed Jun. 11, 2019, and to U.S. Provisional Patent Application No. 62/926,204 entitled “ANTIMICROBIAL QUANTUM DOTS,” filed Oct. 25, 2019, the disclosures of which are incorporated herein by reference in their entireties.

### BACKGROUND OF THE INVENTION

Multidrug resistant pathogens are critical impending problem in global health. In a 2014 commissioned study on antimicrobial resistance, the impact was estimated to be upward of 300 million lives and $100 trillion by 2050. More recently, this mounting risk prompted the World Health Organization (WHO) to release a first-ever list of “priority pathogens”, classified by three tiers of urgency: critical, high, medium. The highest tier corresponds to existing pathogens with resistance to carbapenem (a last resort antibiotic) and includes strains of Escherichia coli.

Antibiotic resistance is compounded by a steady decline in new antibiotics entering the market, growing resistance in pathogens against traditional small molecule therapies, and a lack of financial incentives and scientific breakthroughs in designing a new class of antibiotics. There exists a need in the art to develop therapeutic compositions that combat multidrug resistant pathogens and offer advantages such as stability, facile transport, low cost, and scalable syntheses. The present disclosure address this need.

### BRIEF SUMMARY OF THE DISCLOSURE

In certain embodiments, the disclosure provides a composition comprising at least one indium phosphide (InP) quantum dot or ternary zinc cadmium telluride (Zn₁₋ₓCdₓTe) quantum dot. In certain embodiments, the InP quantum dot or the Zn₁₋ₓCdₓTe quantum dot has a conduction band position above about −0.33 eV, as referenced to normal hydrogen electrode (NHE), at pH 7. In certain embodiments, the InP quantum dot or the Zn₁₋ₓCdₓTe quantum dot has a valence band position below about +1.0 eV, as referenced to NHE at pH 7. In certain embodiments, x a number greater than 0 and less than 1.

In certain embodiments, the disclosure provides a method of killing, preventing, or hampering the growth of a first cell by irradiating the composition presented herein with electromagnetic radiation in the presence of a first cell such that the irradiation generates a therapeutically effective amount of superoxide radicals that kill the first cell and/or prevents or hampers the growth of the first cell.

In certain embodiments, the disclosure provides a method a method of killing bacteria, and/or preventing or hampering bacterial growth, in a subject in need thereof, wherein the method comprises steps of administering to the subject a therapeutically effective amount of the composition presented herein; and irradiating the quantum dot to generate a therapeutically effective amount of superoxide radical that kills, and/or prevents growth, and/or hampers growth of the bacteria.

### BRIEF DESCRIPTION OF THE DRAWINGS

The following detailed description of specific embodiments of the disclosure will be better understood when read in conjunction with the appended drawings. For the purpose of illustrating the disclosure, exemplary embodiments are shown in the drawings. It should be understood, however, that the disclosure is not limited to the precise arrangements and instrumentalities of the embodiments shown in the drawings.

FIGS. 1A-1E illustrate optoelectronic and photoelectrochemical properties of designed InP QDs. FIG. 1A shows absorption spectra for InP QDs (quantum dots) absorbing red and NIR (near-IR) light. Both QDs are water soluble and stabilized with negatively-charged MPA (3-mercaptopropionic acid), and are smaller than 6 nm in diameter. FIG. 1B shows absorption Photoluminescence spectra for “Red InP” showing low energy emission with a tail into NIR. “NIR InP” is nonfluorescent (inset). FIG. 1C is a schematic depicting the biological optical window in relation to InP QD light activation. Light with wavelengths between 650 and 950 nm can penetrate deepest (several centimeters). FIG. 1D shows CB/VB positions for InP QDs, as measured by DPV. Both show narrow bandgaps and potential to generate superoxide. FIG. 1E is EPR spectra for light-activated InP QDs demonstrating superoxide production.

FIGS. 2A-2B illustrate the therapeutic effect of InP QDs in E. coli. The growth curves of E. coli treated with Red InP (FIG. 2A) and NIR InP (FIG. 2B) in the presence of light are shown.

FIGS. 3A-3C illustrate a therapeutic window for nanotherapy, according to some embodiments. Bacterial susceptibility to superoxide nanotherapy at the time of saturated E. coli growth (FIG. 3A), relative to human cells (FIG. 3B), provides a useful therapeutic window of dosage (FIG. 3C). This window of dosage corresponds to QD concentration which effectively kills greater than 99.5% of bacteria (Equations 2 and 3), but is nontoxic to human cells. Human cell toxicity of both QDs is as measured.

FIGS. 4A-4B illustrate that InP QDs can kill MDR priority I pathogens. The growth curve of multidrug-resistant E. coli treated with Red InP (FIG. 4A) and NIR InP (FIG. 4B) in the presence of light is shown.

FIG. 5 shows differential pulse voltammetry measurements. Electrochemical properties were determined using DPV. Spectra could be used for determining CB and VB positions f or Red InP (left) and NIR InP (right).

FIG. 6 are light emission spectra for red LED lamp used in experiments. Emission spectra shows clear peak above 600 nm.

FIGS. 7A-7D are graphs showing relative extinction coefficients and peak for skin and hemoglobin versus InP quantum dots. FIG. 7A shows that human tissue absorbs more light in higher energy wavelengths, with some minimal absorption in red and beyond. FIG. 7B shows that for InP QDs, peak absorption occurs in the red/NIR wavelengths, where absorption from tissue is minimal. FIGS. 7C-7D show the extinction coefficients of oxygenated hemoglobin (HbO₂) and deoxygenated hemoglobin (Hb) plotted on a log scale. Absorption for the components decreases into the near-IR range, where light from 550 nm (yellow shading) will be absorbed much greater than light at 650 nm (red) or 740 nm (dark red).

FIG. 8 is a graph showing an optical model of monochromatic light penetration through various tissue to sufficiently activate therapeutic InP QDs. Deep red (650 nm) and near-IR (740 nm) light can penetrate more deeply than visible light, such as 550 nm. Absorption through the skin is a barrier for 550 nm light, but longer wavelengths can permeate to reach deeper layers. Optical absorbance of breast, bone, lung tissue for red, near-IR light all allow for several centimeters of light penetration. Results presented here are based on 120 mW/cm² monochromatic light and nontoxic bactericidal QD dosage.

FIGS. 9A-9B show graphs of photo-degradation of InP QDs.

FIGS. 10A-10F are microscope images of HeLa after nanotherapy treatment with bactericidal dose. (FIGS. 10A, 10D) No treatment control. Treatment with 500 nM Red InP (FIGS. 10B, 10E) and 1000 nM of NIR InP (FIGS. 10C, 10F) results in negligible toxicity or morphological changes. Flat cells appearing with low contrast correspond to healthy, well-adhered cells. Circular, higher-contrast cells correspond to less tightly-adhered cells—this effect is not exclusive to treatment, as the “No Treatment” control can and does contain a small amount of circular cells. A low cell density or absence of cells corresponds to cell death-nonviable cells which have fully detached from the surface. Scale bars represent 1000 μm (FIGS. 10A-10C), and 100 μm (FIGS. 10D-10F).

FIGS. 11A-11D show microscope images of HeLa cells after nanotherapy treatment InP QDs. Treatment with an excess dose of 1000 nM Red InP (FIGS. 11A, 11C) and 2000 nM of NIR InP (FIGS. 11B, 11D) results in slight signs of toxicity, and some morphological changes in Red InP. Scale bars represent 1000 μm (FIGS. 11A, 11B), and 100 μm (FIGS. 11C, 11D).

FIG. 11E is a graph showing the viability of cells upon treatment with doses described in FIGS. 11A-11D using a standard resazurin assay.

FIGS. 12A-12C illustrate various properties of InP quantum dots. FIG. 12A is a graph illustrating InP quantum dot particle size as measured by dynamic light scattering (DLS). FIG. 12B shows bandgaps in Red InP and NIR Inp quantum dots and FIG. 12C summarizes InP size, bandgap, and conduction band position for InP quantum dots, according to some embodiments.

FIG. 13 is a photograph of solutions containing red-absorbing Red InP (left) and near-IR absorbing NIR InP (right).

FIGS. 14A-14B are EPR spectra showing evolution of radical adduct signal with time. FIG. 14A is a fitted spectrum immediately after illumination showing clear signs of superoxide (DMPO-OOH; green arrows, bottom trace). As time progresses, this radical adduct decays, releasing hydroxyl radicals, which then form DMPO-OH (blue arrows). FIG. 14B is a spectrum of residuals plotted to show the accuracy of the fitted signal in FIG. 14A.

FIGS. 15A-15C illustrate delayed growth and reduced growth rate upon treatment with InP QDs. FIG. 15A shows that reducing the initial cell dilution 10-fold causes an extension in lag phase (left) but does not significantly affect the growth rate of E. coli. FIG. 15B shows that InP QDs result in delayed growth, or extension of the apparent lag phase, while causing a bactericidal effect (105 dilution). FIG. 15C shows the growth rate (μ) of E. coli under the influence of a control, Red InP, and NIR InP.

FIGS. 16A-16F illustrate design of ternary Zn₁₋ₓCdₓTe QDs. FIG. 16A shows absorption/photoluminescence spectra (solid/dashed) for CdTe-2.4 and ZnTe on respective scales. FIG. 16B shows CB-VB positions (vs. NHE) of CdTe-2.4 and ZnTe QDs compared to redox potentials for water oxidation/reduction reactions (dashed lines). FIG. 16C shows EPR spectra of ZnTe QDs illuminated with UV light (top) and CdTe-2.4 QDs illuminated with visible light (bottom), confirming superoxide generation. FIGS. 16D and 16E show normalized growth curves for E. coli MG1655 in the presence and absence of photoactivated CdTe (FIG. 16D) and ZnTe (FIG. 16E) in LB medium. The error bars, spanning two standard deviations, represent three biological replicates. FIG. 16F is a schematic illustrating depicting visible light activity of CdTe-2.4 (left), UV requirement for ZnTe (middle), and proposed visible light active ternary Zn₁₋ₓCdₓTe QDs (right).

FIGS. 17A-17C illustrate tuning the bandgap, electronic states, and CB-VB position in Zn₁₋ₓCdₓTe QDs. FIG. 17A shows the breakdown of cation composition in Zn₁₋ₓCdₓTe QDs obtained from different synthetic conditions. The error bars, spanning two standard deviations, represent three biological replicates. FIG. 17B shows photoluminescence spectra for Zn₁₋ₓCdₓTe of varying composition. FIG. 17C shows CB-VB positions (vs. NHE) of QDs showing that superoxide generation occurs. Darker shades of blue denote greater zinc incorporation.

FIGS. 18A-18C illustrate that Zn₁₋ₓCdₓTe displays strong photoinhibition by generating intracellular superoxide. FIG. 18A shows quantification of superoxide generated by Zn₁₋ₓCdₓTe suspensions after visible light illumination during EPR spectroscopic measurement. Darker shades of blue denote greater zinc incorporation. FIG. 18B shows normalized growth curves for E. coli MG1655 in the presence and absence of photoactivated Zn₁₋ₓCdₓTe. Equal concentrations (12.5 nM) of each ternary QDs were used in in vitro bacterial cell culture tests. FIG. 18C shows inhibition of MG1655 at t=12 h using a 12.5 nM dose of ternary QDs. The error bars, spanning two standard deviations, represent three biological replicates.

FIGS. 19A-19C illustrate that Zn₁₋ₓCdₓTe kills priority 1 pathogen with minimal cadmium and shows no toxicity in mammalian cells. FIG. 19A shows the effective cadmium content calculated using the product of GIC₅₀ and parts per billion (ppb) of cadmium content of the QDs for the different Zn₁₋ₓCdₓTe QDs. Zn_{0.63}Cd_{0.37}Te (*) requires the least cadmium to inhibit bacterial growth by 50%. Data are plotted with known ranges of cadmium content from common consumable sources. FIG. 19B shows LDH toxicity assay results for photoactivated Zn_{0.63}Cd_{0.37}Te. FIG. 19C shows normalized growth curve for CRE E. coli in the presence and absence of photoactivated Zn_{0.63}Cd_{0.37}Te. The error bars, spanning two standard deviations, represent three biological replicates.

FIG. 20 is a ZnTe Photoluminescence (PL) spectrum.

FIGS. 21A-21B show CdTe-2.4 (FIG. 21A) and ZnTe (FIG. 21B) Differential Pulse Voltammetry measurements. DPV measurements show band positions for the two different QDs.

FIG. 22 shows a series of Zn₁₋ₓCdₓTe absorbance spectra. Increasing zinc composition of Zn₁₋ₓCdₓTe causes a blue shift in excitonic peaks.

FIGS. 23A-23D show EPR spectra of Zn₁₋ₓCdₓTe QDs illustrating superoxide generation upon visible light activation.

FIG. 24 is a graph illustrating determination GIC₅₀ values individually for each QD of varying composition (x). First, inhibition at t=12 hours was calculated using Equation 8. 12 hours as a timepoint was selected that showed equilibrated growth in no-treatment control. Because discrete values of QD dosage (12.5, 25, 50, 100 nM) were tested, each GIC₅₀ was selected as the minimum dosage which demonstrated at least 50% inhibition.

FIGS. 25A-25B illustrate that Zn₁₋ₓCdₓTe QDs show negligible HeLa cell toxicity.

FIG. 25A shows 10× magnification images of HeLa cell growth after 18 hours of no treatment condition (negative control). FIG. 25B shows 10× magnification images of HeLa cells treated with 50 nM Zn_{0.63}Cd_{0.37}Te QDs. Cells appear adherent and healthy compared to positive and negative controls. The scale bars show 400 m. All conditions were exposed to the same light source for the same duration.

FIG. 26 is an emission spectra for LED sheet used to activate quantum dots during in vitro experiments.

FIGS. 27A-27C illustrates a variety of Zn₁₋ₓCdₓTe QD physical and electronic properties. FIG. 27A shows particle diameter measurement using dynamic light scattering.

FIG. 27B shows particle diameters as a function of ‘x’ and FIG. 27C is a table showing values for bandgap and position of the conduction band for different compositions of Zn₁₋ₓCdₓTe quantum dots.

FIGS. 28A-28F are transmission electron microscopy images of Zn₁₋ₓCdₓTe QDs.

FIG. 28A, x=0.96. FIG. 28B, x=0.89. FIG. 28C, x=0.37. FIG. 28D, x=0.32. FIG. 28E, close-up image of therapeutic x=0.37 ternary QDs. FIG. 28F is a table showing the measured diameters for each ternary QD of varying composition. Scale bar represents 10 nm.

FIGS. 29A-29B illustrate LDH assay of Zn_{0.63}Cd_{0.37}Te QDs in multiple human cell lines. Lactate Dehydrogenase assay for QDs in liver cells (HepG2-C3A; FIG. 29A) and osteoblasts (MC3T3-E1; FIG. 29B). HepG2-C3A were seeded at Passage 10, in DMEM with 10% FBS; MC3T3-E1 were seeded at Passage 10, in MEM with 10% FBS. None of the treatment conditions showed statistically significant toxicity versus the negative control.

### DETAILED DESCRIPTION OF THE INVENTION

The present disclosure relates to developing superoxide-generating QDs as selective therapeutics to counter the growing threat of antimicrobial-resistant infections. The process for rational design of superoxide-generating QDs relies on having a small bandgap (for light activation), a sufficiently high CB position to donate electrons to dissolved oxygen and produce superoxide selectively, a VB position low enough to prevent generation of indiscriminately reactive hydroxyl radical on light activation, and appropriate QD surface and ligand charge to ensure a small hydrodynamic radius for facile transport, uptake by cells, and ultimate clearance by the body. Accordingly, considering all the above enlisted parameters, InP-based and Zn₁₋ₓCdₓTe (where, 0<x<1) based quantum dots have been developed herein. Further, experimental examples have been presented herein to demonstrate the effectiveness of these quantum dots to counter the multidrug-resistant bacteria.

### Definitions

Unless defined otherwise, all technical and scientific terms used herein have the same meaning as commonly understood by one of ordinary skill in the art to which the disclosure pertains. Although any methods and materials similar or equivalent to those described herein can be used in the practice for testing of the present disclosure, selected materials and methods are described herein. In describing and claiming the present disclosure, the following terminology will be used.

It is also to be understood that the terminology used herein is for the purpose of describing particular embodiments only, and is not intended to be limiting.

As used herein, the articles “a” and “an” refer to one or to more than one (i.e. to at least one) of the grammatical object of the article. By way of example, “an element” means one element or more than one element.

As used herein, the term “about” will be understood by persons of ordinary skill in the art and will vary to some extent on the context in which it is used. As used herein, “about” when referring to a measurable value such as an amount, a temporal duration, and the like, is meant to encompass variations of ±20% or ±10%, more specifically ±5%, even more specifically ±1%, and still more specifically ±0.1% from the specified value, as such variations are appropriate to perform the disclosed methods.

As used herein, the term “band gap” refers to distance between the valence band of electrons and the conduction band. Essentially, the band gap represents the minimum energy that is needed to excite an electron up to a state in the conduction band where it can participate in conduction.

As used herein, the term “conduction band” refers to the band of electron orbitals that electrons can jump up into from the valence band when excited. When the electrons are in these orbitals, they have enough energy to move freely in the material. This movement of electrons creates an electric current.

As used herein, the term “Red InP” refers to InP quantum dots that strongly absorb light having wavelength of about 650 nm to about 700 nm.

As used herein, the term “Red InP” refer to InP quantum dots that strongly absorb light having wavelength of about 700 nm to about 1400 nm.

The terms “patient,” “subject” or “individual” are used interchangeably herein, and refer to any animal, or cells thereof whether in vitro or in situ, amenable to the methods described herein. In a non-limiting embodiment, the patient, subject or individual is a human. In other embodiments, the patient is a non-human mammal including, for example, livestock and pets, such as ovine, bovine, porcine, canine, feline and murine mammals. In yet other embodiments, the patient is an avian animal or bird. Preferably, the patient, individual or subject is human.

As used herein, the term “therapeutically effective amount” refers to an amount that is sufficient or effective to prevent or treat (delay or prevent the onset of, prevent the progression of, inhibit, decrease or reverse) a disease or condition described or contemplated herein, including alleviating symptoms of such disease or condition.

As used herein, the term “heavy metals” refers to metals with relatively high densities, atomic weights, or atomic numbers, and having a specific gravity of 5.0 or greater. Examples of heavy metals include, but are not limited to, cobalt (Co), copper (Cu), chromium (Cr), iron (Fe), magnesium (Mg), manganese (Mn), molybdenum (Mo), nickel (Ni), selenium (Se), Indium (In) and zinc (Zn).

As used herein, the term “treatment” or “treating” is defined as the application or administration of a therapeutic agent, i.e., a compound of the disclosure (alone or in combination with another pharmaceutical agent), to a patient, or application or administration of a therapeutic agent to an isolated tissue or cell line from a patient (e.g., for diagnosis or ex vivo applications), who has a condition contemplated herein, a symptom of a condition contemplated herein or the potential to develop a condition contemplated herein, with the purpose to cure, heal, alleviate, relieve, alter, remedy, ameliorate, improve or affect a condition contemplated herein, the symptoms of a condition contemplated herein or the potential to develop a condition contemplated herein. Such treatments may be specifically tailored or modified, based on knowledge obtained from the field of pharmacogenomics

As used herein, the term “valence band” is the band of electron orbitals that electrons can jump out of, moving into the conduction band when excited. The valence band is simply the outermost electron orbital of an atom of any specific material that electrons actually occupy.

Throughout this disclosure, various aspects may be presented in a range format. It should be understood that the description in range format is merely for convenience and brevity and should not be construed as an inflexible limitation on the scope of the disclosure. Accordingly, the description of a range should be considered to have specifically disclosed all the possible sub-ranges as well as individual numerical values within that range and, when appropriate, partial integers of the numerical values within ranges. For example, description of a range such as from 1 to 6 should be considered to have specifically disclosed sub-ranges such as from 1 to 3, from 1 to 4, from 1 to 5, from 2 to 4, from 2 to 6, from 3 to 6, and so on, as well as individual numbers within that range, for example, 1, 2, 2.7, 3, 4, 5, 5.3, and 6. This applies regardless of the breadth of the range.

The following abbreviations are used herein: VB=valance band; CB=conduction band; MPA=3-mercaptopropionic acid; EPR=electron paramagnetic resonance; QD=quantum dot; PL=Photoluminescence; GIC₅₀=growth inhibition concentration; MIC=minimum inhibitory concentrations; OD=optical density; DPV=Differential pulse voltammetry; MDR=multidrug-resistant; LDH=lactate dehydrogenase.

### Compositions

In certain embodiments, the disclosure provides a composition comprising at least one indium phosphide (InP) quantum dot or a ternary zinc cadmium telluride (Zn₁₋ₓCdₓTe) quantum dot. In certain embodiments, x is greater than 0 and less than 1. In certain embodiments, the quantum dot has a conduction band position above about −0.33 eV, as referenced to NHE (normal hydrogen electrode) at pH 7. In certain embodiments, the quantum dot has a valence band position below about +1.0 eV, as referenced to NHE, at pH 7. In certain embodiments, the quantum dot is capable of generating superoxide radical upon irradiation with a light of suitable wavelength by donating electron(s) to the dissolved oxygen.

In certain embodiments, the quantum dot absorbs light and triggers the photo-electrochemical reduction of oxygen to selectively generate superoxide. In certain embodiments, the quantum dots presented herein do not generate any other reactive oxygen species (ROS) such as, for example, hydroxyl radical, (OH.), hydrogen peroxide (H₂O₂), and singlet oxygen (¹O₂) which have very different roles and reactivity in cells.

Since longer wavelengths of light penetrate deeper through human tissue, owing to the optical window of biological transparency for light with wavelengths of about 650 nm to 1350 nm, it is desirable to create heavy metal-free therapeutic QDs triggered by near-IR light, such as, for example, InP quantum dots as described herein.

In certain embodiments, the composition comprising InP quantum dots is completely devoid of traces of heavy metals. In certain embodiments, the concentration of traces of heavy metals in the composition comprising InP quantum dots is less than about 0.001% to about 1%.

In certain embodiments, wherein the InP quantum dot is 2 nm to about 7 nm in diameter. In certain embodiments, the diameter of InP quantum dot is about 2.0, 2.2, 2.4, 2.6, 2.8, 3.0, 3.2, 3.4, 3.6, 3.8, 4.0, 4.2, 4.4, 4.6, 4.8, 5.0, 5.2, 5.4, 5.6, 5.8, 6.0, 6.2, 6.4, 6.6, 6.8 or about 7.0 nm.

In certain embodiments, varying the diameter of the quantum dot varies its optoelectronic and redox properties. For example, in one specific embodiment, when the diameter of the InP quantum dot is about 5 nm to about 7 nm, the InP quantum dot (referred to, in some embodiments, as Red InP) has a strong absorbance at a wavelength of about 650 nm with an excitonic peak around 575 nm and an emission peak centered around 601 nm, whereas in another embodiment, when the diameter of the InP quantum dot is about 4 nm to about 7 nm, InP is a near-infrared (NTR) InP and has a strong absorbance at a wavelength about 700 nm with an excitonic peak around 720 nm.

In certain embodiments, the conduction band position of an InP quantum dot is about −0.9 eV to about −1.2 eV, as referenced to NHE (standard hydrogen electrode) at pH 7. In certain embodiments, the conduction band position of an InP quantum dot is about −0.90, −0.92, −0. 94, −0.96, −0.98, −1.0, or about −1.2 eV. In one specific embodiment, the conduction band position of InP quantum dot is about −1.052 eV.

In certain embodiments, the conduction band position of a NIR InP (near infrared) quantum dot is about −0.5 eV to about −0.8 eV, as referenced to NHE (standard hydrogen electrode) at pH 7. In certain embodiments, the conduction band position of NIR InP is about −0.5, −0.52, −0.54, −0.56, −0.58, 0.60, −0.62, −0.64, −0.66, −0.68, −0.70, −0.72, −0.74, −0.76, −0.78, or about −0.8. In one specific embodiment, the conduction band position of the NIR InP quantum dot is about −0.71 eV.

In certain embodiments, when the at least one nanoparticle comprises InP quantum dot, the at least one nanoparticle is at least partially coated with one selected from the group consisting of ZnS and ZnSe. Coating with ZnS and ZnSe enhances chemical stability and biocompatibility of the quantum dots. In certain embodiments, the quantum dots are water-soluble. The quantum dots described herein can be, in some embodiments, at least partially coated by 3-mercaptopropionic acid (MPA).

In certain embodiments, the InP quantum dot has a band gap of about 0.9 eV to about 1.9 eV. In certain embodiments, the InP quantum dot has a band gap of about 0.9, 1.10, 1.15, 1.20, 1.25, 1.30, 1.35, 1.40, 1.45, 1.50, 1.55, 1.60, 1.65, 1.70, 1.75, 1.80, 1.85, or about 1.90 eV. In one specific embodiment, the InP quantum dot has a band gap of about 1.35 eV.

In certain embodiments, the ternary zinc cadmium telluride (Zn₁₋ₓCdₓTe) are compositionally tunable and the x has a value of about 0.98 to about 0.25. In certain embodiments, the x has a value of about 0.98, 0.95, 0.90, 0.85, 0.80, 0.75, 0.70, 0.65, 0.60, 0.55, 0.50, 0.45, 0.40, 0.35, 0.30, or about 0.25. In one specific embodiment, the x is about 0.96. In another specific embodiment, x is about 0.89. In yet another specific embodiment, x is about 0.37. In yet another specific embodiment, x is about 0.32. In various embodiments, x is about 0.3, 0.31, 0.32, 0.33, 0.34, 0.35, 0.36, 0.37, 0.38, 0.39, 0.40, 0.41, 0.42, 0.43, 0.44, or 0.45. In some embodiments, x is about 0.35 to about 0.45.

In certain embodiments, the diameter of Zn₁₋ₓCdₓTe quantum dot is about 2.9 nm to about 3.9 nm. In certain embodiment, the diameter of Zn₁₋ₓCdₓTe quantum dot is about 2.9, 3.0, 3.1, 3.2, 3.3, 3.4, 3.5, 3.6, 3.7, 3.8, or about 3.9 nm.

In certain embodiments, the Zn₁₋ₓCdₓTe quantum dot has a band gap of about 2.3 eV to about 3.3 eV. In certain embodiments, the Zn₁₋ₓCdₓTe quantum dot has a band gap of about 2.3, 2.4, 2.5, 2.6, 2.7, 2.8, 2.9, 3.0, 3.1, 3.2 or about 3.3 eV.

In one specific embodiment, the Zn_{0.63}Cd_{0.37}Te quantum dot and has a conduction band position of about 1.0 eV to about 1.1 eV, as referenced to NHE (standard hydrogen electrode) at pH 7. In certain embodiments, the Zn_{0.63}Cd_{0.37}Te quantum dot has a bulk band gap of about 2.5 eV to about 2.7 eV.

In certain embodiments, the composition presented herein are acutely toxic to bacteria, and nontoxic to mammalian cells. In certain embodiments, the composition is nontoxic to human cells. In various embodiments, the InP quantum dot is the only quantum dot present in the composition. In various embodiments, the Zn₁₋ₓCdₓTe quantum dot is the only quantum dot present in the composition.

### Methods

In another embodiment, the disclosure provides a method of killing, or preventing or hampering growth, of a first cell, wherein the method comprises irradiating the quantum dot compositions described herein with electromagnetic radiation, in the presence of a first cell, wherein the irradiation generates a therapeutically effective amount of superoxide radicals that kills the first cell, or prevents or hampers the growth of the first cell.

In certain embodiments, the disclosure provides a method of killing, or preventing, or hampering bacterial growth in a subject in need thereof, wherein the method includes administering to the subject a therapeutically effective amount of a composition comprising quantum dots described herein, wherein irradiating the quantum dot(s) with electromagnetic radiation generates a therapeutically effective amount of superoxide radical that effects killing, or preventing or hampering growth, of the bacteria without causing any measurable effect on the cells of the subject. In certain embodiments, the composition and the quantum dots are as described elsewhere herein. Electromagnetic radiation suitable for use in irradiating the quantum dot compositions described herein includes light having the wavelengths described herein.

In certain embodiments, when the quantum dot is InP, the wavelength of the electromagnetic radiation to generate the superoxide ranges from about 650 nm to about 1000 nm. In certain embodiments, when the quantum dot is InP, the irradiation wavelength to generate the superoxide is selected from the group consisting of about 650, 700, 750, 800, 850, 900, 950, and about 1000 nm.

In certain other embodiments, when the quantum dot is Zn₁₋ₓCdₓTe the wavelength of the electromagnetic radiation to generate superoxide ranges from about 400 nm to about 700 nm. In certain embodiments, the Zn₁₋ₓCdₓTe quantum dot generates superoxide upon irradiation with a visible the light having a wavelength selected from the group consisting of 400, 450, 500, 550, 600, 650, and about 700 nm.

In certain embodiments, the first cell is a bacterium. In certain embodiments, the first cell is a multidrug-resistant bacterium. In certain embodiments, the bacterium is a multidrug-resistant bacterium. In certain embodiments, the bacterium comprises at least one selected from the group consisting of Enterobacteriaceae E. coli (CRE E. coli), Mycobacterium tuberculosis, K. pneumonia, E. coli, S. aureus, P. aeruginosa, A. baumannii and S. typhimurium.

In certain embodiments, the first cell comprises a Gram-negative bacterium, which is further contacted with at least one Gram-negative antibacterial agent. In certain embodiments, the concentration or amount of the antibacterial agent that is required to kill, or prevent or hamper the growth of, the first cell in the presence of the at least one quantum dot is lower than the concentration or amount of the antibacterial agent that is required to kill, or prevent or hamper the growth of, the first cell when the antibacterial agent is used in the absence of the at least one quantum dot. Suitable antibacterial agents for use with the quantum dots described herein include agents that inhibit cell wall synthesis such as penicillin, ampicillin, amoxicillin and the like; agents that inhibit bacterial protein synthesis such as aminoglycosides (e.g., gentamicin, tobramycin, amikacin), macrolides (e.g., erythromycin, clarithromycin, azithromycin), tetracylines (e.g., Tetracycline, Doxycycline), Chloramphenicol, lincomycins (e.g., clindamycin); and agents that inhibit bacterial nucleic acid synthesis such as sulfonamides (e.g., sulfisoxazole, sulfamethoxazole), metronidazole, pyrimidine derivatives, rifampicin, quidnolones. Other antibacterial agents known to those of skill in the art can also be used with the quantum dots described herein.

In certain embodiments, the first cell is in the presence of a second cell, and wherein irradiation of the first and second cell in the presence of the at least one nanoparticle has no measurable effect on the growth, metabolism or survival of the second cell.

In certain embodiments, the second cell is mammalian cell. In certain embodiment, the second cell is a human cell.

In certain embodiments, the subject is a mammal. In certain embodiment, the subject is a human.

### Administration/Dosage/Formulations

The regimen of administration may affect what constitutes an effective amount. The therapeutic formulations may be administered to the subject either prior to or after the onset of a bacterial infection. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions described herein to a patient, preferably a mammal, more preferably a human, may be carried out using known procedures, at dosages and for periods of time effective to treat a bacterial infection in the patient. An effective amount of the therapeutic compound necessary to achieve a therapeutic effect may vary according to factors such as the state of the disease or disorder in the patient; the age, sex, and weight of the patient; and the ability of the therapeutic compound to treat a bacterial infection in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound described herein is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions described herein may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds described herein employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the compound(s) described herein are dictated by and directly dependent on (a) the unique characteristics of the therapeutic compound and the particular therapeutic effect to be achieved, and (b) the limitations inherent in the art of compounding/formulating such a therapeutic compound.

In certain embodiments, the compositions described herein are formulated using one or more pharmaceutically acceptable excipients or carriers. In certain embodiments, the pharmaceutical compositions described herein comprise a therapeutically effective amount of a compound described herein and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions described herein are administered to the patient in dosages that range from one to five times per day or more. In other embodiments, the compositions described herein are administered to the patient in range of dosages that include, but are not limited to, once every day, every two, days, every three days to once a week, and once every two weeks. It is readily apparent to one skilled in the art that the frequency of administration of the various combination compositions described herein varies from individual to individual depending on many factors including, but not limited to, age, disease or disorder to be treated, gender, overall health, and other factors. Thus, administration of the compounds and compositions described herein should not be construed to be limited to any particular dosage regime and the precise dosage and composition to be administered to any patient is determined by the attending physician taking all other factors about the patient into account.

The compound(s) described herein for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 350 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound described herein is from about 1 mg and about 2,500 mg. In some embodiments, a dose of a compound described herein used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

Routes of administration of any of the compositions described herein include oral, nasal, rectal, intravaginal, parenteral, buccal, sublingual or topical. The compounds for use in the compositions described herein can be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compound(s) described herein can be in the form of tablets or capsules prepared by conventional means with pharmaceutically acceptable excipients such as binding agents (e.g., polyvinylpyrrolidone, hydroxypropylcellulose or hydroxypropyl methylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Compositions as described herein can be prepared, packaged, or sold in a formulation suitable for oral or buccal administration. A tablet that includes a compound as described herein can, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, dispersing agents, surface-active agents, disintegrating agents, binding agents, and lubricating agents.

Parenteral Administration

For parenteral administration, the compounds as described herein may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1, 3-butanediol. Among the acceptable vehicles and solvents that may be employed are water, Ringer's solution and isotonic sodium chloride solution. Sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as Ph. Helv or similar alcohol.

Additional Administration Forms

Additional dosage forms suitable for use with the compound(s) and compositions described herein include dosage forms as described in U.S. Pat. Nos. 6,340,475; 6,488,962; 6,451,808; 5,972,389; 5,582,837; and 5,007,790. Additional dosage forms suitable for use with the compound(s) and compositions described herein also include dosage forms as described in U.S. Patent Applications Nos. 20030147952; 20030104062; 20030104053; 20030044466; 20030039688; and 20020051820. Additional dosage forms suitable for use with the compound(s) and compositions described herein also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

Those skilled in the art will recognize, or be able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

The practice of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Animal Cell Culture” (Freshney, 1987); “Methods in Enzymology” Handbook of Experimental Immunology” (Weir, 1996). Particularly useful techniques for particular embodiments will be discussed in the sections that follow.

It is to be understood that, wherever values and ranges are provided herein, the description in range format is merely for convenience and brevity and should not be construed as an inflexible limitation on the scope of the disclosure. Accordingly, all values and ranges encompassed by these values and ranges are meant to be encompassed within the scope of the present disclosure. Moreover, all values that fall within these ranges, as well as the upper or lower limits of a range of values, are also contemplated by the present application. The description of a range should be considered to have specifically disclosed all the possible sub-ranges as well as individual numerical values within that range and, when appropriate, partial integers of the numerical values within ranges. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The following examples further illustrate aspects of the present disclosure. However, they are in no way a limitation of the teachings or disclosure of the present disclosure as set forth herein.

### Experimental Examples

The disclosure is further described in detail by reference to the following experimental examples. These examples are provided for purposes of illustration only, and are not intended to be limiting unless otherwise specified. Thus, the disclosure should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present disclosure and practice the claimed methods. The following working examples therefore, specifically point out the preferred embodiments of the present disclosure, and are not to be construed as limiting in any way the remainder of the disclosure.

### Experimental Procedures

### InP Quantum Dots

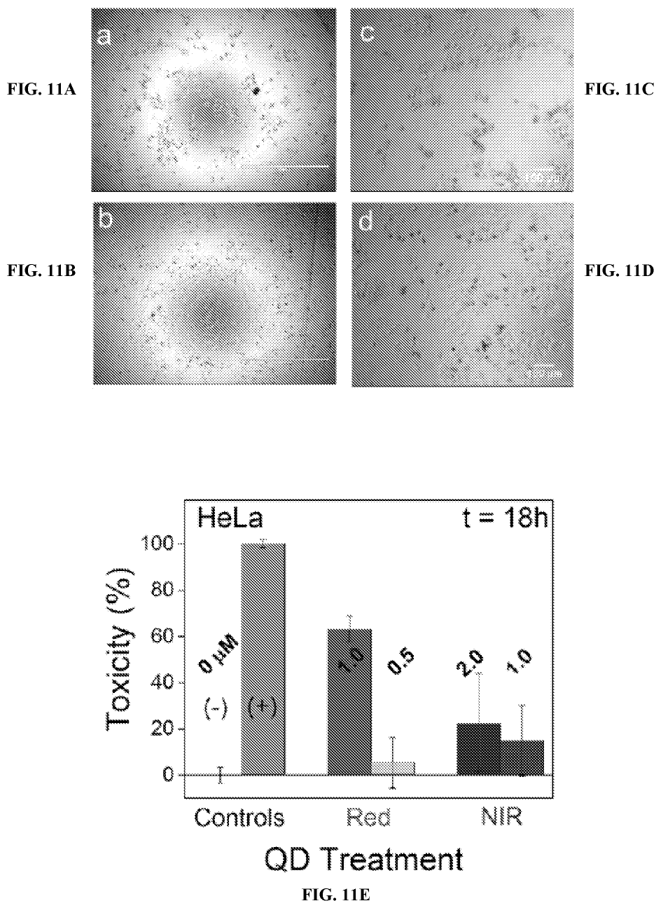

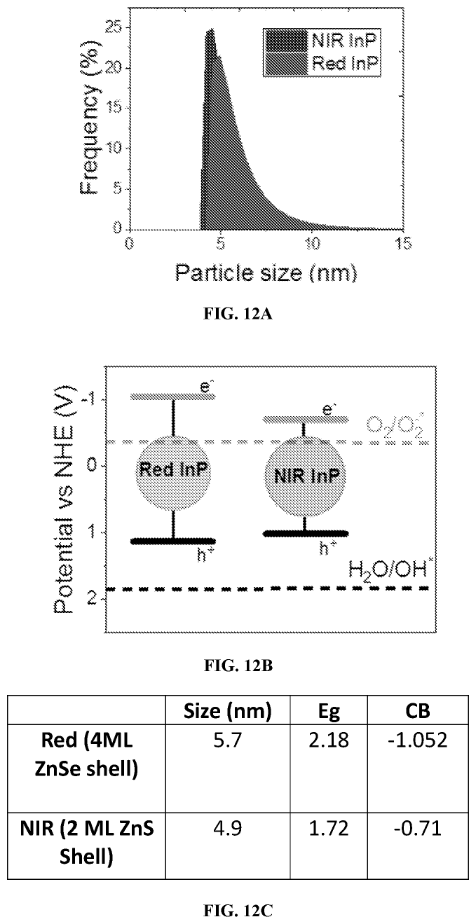

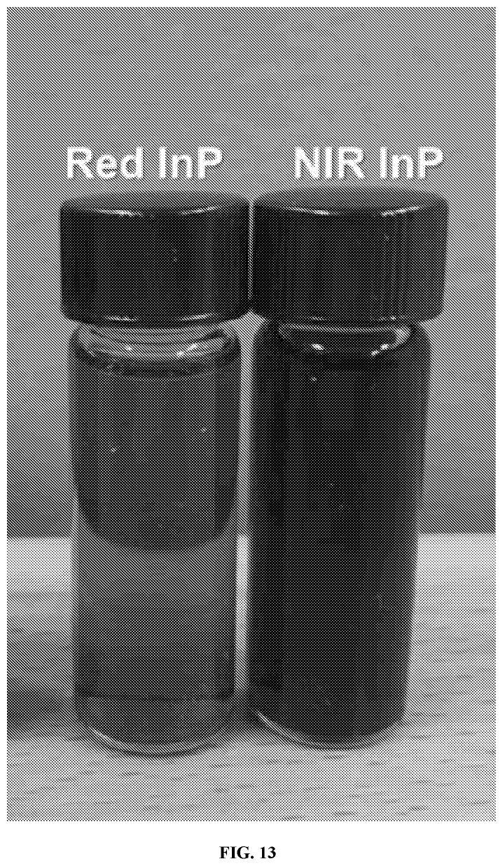

InP QD synthesis was adapted from Tessier et al. (Tessier, M. D. et al., 2015, Chemistry of Materials, 27(13), 4893-4898). Briefly, indium (III) chloride, zinc chloride, and hexamethylphosphorous triamide were reacted in oleylamine to form InP cores in the organic phase, before shell growth. QDs were then transferred to the aqueous phase using 3-mercaptopropionic acid as a hydrophilic ligand (FIG. 13). Superoxide radicals were detected via spin-trap EPR spectroscopy using a Bruker Elexsys E500 spectrometer and DMPO (FIGS. 12A-12C). Cell-killing was assessed using bacterial cell culture in Lysogeny broth (LB) medium by tracking optical density in a Tecan GENios plate reader. The CRE E. coli clinical isolate was kindly provided by the University of Colorado's Anschutz Medical Campus. This strain was cultured in cation adjusted Mueller Hinton broth. HeLa cells were maintained in Dulbecco's minimal essential media with 10% fetal bovine serum and penicillin/streptomycin at 37° C., 5% CO₂, and controlled humidity. Toxicity to human cells was assessed via resazurin assay of HeLa cells seeded on a 96-well plate after treatment (QD) or control (negative: no treatment; positive: Triton X-100). All error bars span two standard deviations. More methodological details are provided elsewhere herein.

### Synthesis of InP QDs of Varying Absorbance

The synthesis method used for InP QDs of varying absorbance was adapted from Tessier, et al. for our purposes. For the “Red InP” quantum dot cores, indium(III) chloride (InCl₃, 100 mg) and zinc(II) chloride (ZnCl₂, 300 mg) were added to 5 mL oleylamine (OLA) in a two-neck flask with magnetic stirring. The mixture was degassed for 2 hours at 120° C., and subsequently heated to 180° C. under an argon atmosphere. Once the desired temperature was reached, hexamethylphosphorous triamide (HMPT, 0.45 mL) was swiftly injected into the reaction mixture, initiating InP seed formation and consequent nanocrystal growth. Nanocrystal absorbance was monitored using a VWR UV-1600PC UV/VIS spectrophotometer. Once the desired absorption wavelength of the InP cores was reached, the shell growth process was initiated. To create a ZnSe shell, 1 mL of 2.2 M elemental selenium dissolved in trioctylphosphine (TOP-Se) was slowly injected. After 30 minutes, the temperature was increased to 200° C. and held for 60 additional minutes. Then 1 g of zinc(II) stearate dissolved in 4 mL hot octadecene (ODE) was slowly injected and the temperature increased to 220° C. 30 minutes later, 0.7 mL of 2.2 M TOP-Se was slowly injected and the temperature further increased to 240° C. After another 30 minute period, 0.5 g of zinc(II) stearate in 2 mL ODE is injected slowly followed by increasing the temperature to 260° C. and holding for 30 minutes. The InP/ZnSe nanocrystals were then cooled down to near room temperature by blowing air, precipitated with excess ethanol, and centrifuged to remove excess or unreacted precursors. The nanocrystals were washed several times by resuspension in chloroform or hexane and precipitating with ethanol repeatedly. Finally, the OLA capped InP/ZnSe were stored in hexane or chloroform and kept in the dark. To alternatively create a ZnS shell, 1 mL of 2.2 M elemental sulfur dissolved in trioctylphosphine (TOP-S) was used rather than TOP-Se. To create 720 nm-absorbing “NIR InP,” the same procedure was carried out with two minor modifications. First, ZnCl₂ was omitted from the initial reaction mixture, as it is reported to limit the size of the InP cores during the initial growth stage. Second, the injection temperature of HMPT was increased to 240° C. to allow larger nanocrystals to form.

InP QDs in organic phase were then transferred to aqueous phase via ligand exchange with 3-mercaptopropionic acid (MPA), giving the QDs a negative charge. The OLA-coated nanocrystals were precipitated with ethanol and centrifuging and isolated as a pellet. A small amount of pure MPA was added directly to the pellet, followed by agitation, to redisperse the nanocrystals. Twice the volume of 1M NaOH was added to the mixture and sonicated in a water bath for several minutes. Ethanol was added at a volume equal to 3 times the mixture NaOH, MPA volume and then centrifuged to collect the MPA-coated nanocrystals as a pellet. The nanocrystals were then redispersed in pH 11 water (NaOH) and centrifuged at 5000 rpm for 5 minutes to remove any poorly passivated particles-aggregated QDs or QDs that did not receive sufficient surface coating of MPA. The supernatant containing water-soluble nanocrystals was then stored in the dark at 4° C. In the absence of light, QDs could be stored for weeks or months with no signs of oxidation. For experiments presented here, MPA-QDs were prepared fresh for experiments.

### Quantum Dot Purification and Washing

The bulk of excess reagents should be removed during the ligand exchange process, further steps were taken to prepare the QD suspensions. Aqueous MPA-coated QD suspensions were sterilized, purified and washed before use in any experiment. First, samples were sterilized by transfer to an autoclaved microcentrifuge tube via syringe filter with 220 nm cut-off size. Next, samples were purified by centrifugation in 3 kda Nanosep filters (Pall) at 8000 rpm, for 8:00 minutes. Filtrate was discarded, and retentate was resuspended in pH 11 water for 3 subsequent washing steps. QD concentration could then be determined using UV-vis spectroscopy. Briefly, the QD suspension (aqueous) absorption spectra was collected, and analysed to determine the position of the first excitonic peak (λₚₑₐₖ). An extinction coefficient was determined for InP QDs using that peak position (Equation 1). This extinction coefficient, which corresponds to molar extinction at 310 nm, was then applied to the Beer-Lambert law to determine each QD stock concentration.

$${ɛ}_{310} = 47.27 \times {λ}_{peak ( nm )}^{2} - 3.46 \times {10}^{4} \times {λ}_{peak ( nm )} + 5.8 \; 1 \times 1 {0}^{6} \qquad Equation 1$$

### EPR Spectroscopy

Electron paramagnetic resonance (EPR) spectroscopy was used to detect and identify radical species resulting from photoactivation. The spectrometer, Bruker Elexsys E 500, is equipped with an SHQE resonator operated in a dark room. A microwave attenuation of 16 dB and a power of 5 W was used. Briefly, 90 to 100 μL aliquots of QD suspensions were purified by filtration and washing as described above. 1 μL (1 vol %) of spin-trapping agent 5,5-dimethyl-1-pyrroline N-oxide (DMPO) were added to each sample immediately before measurement. DMPO is required to for detecting radical species, as it forms stable adducts with species such as superoxide and hydroxyl radicals. Samples were wrapped in foil and sequestered entirely from ambient light. Samples suspensions were loaded into three quartz capillaries, sealed, and placed in the EPR tube. A baseline EPR spectrum was measured in the dark conditions. The sample was then removed from the EPR cavity and exposed to 45 seconds of low-energy LED light source (FIG. 6) and swiftly returned to the spectrometer for measurement (estimated time between illumination and measurement ˜ 10 seconds). Unless otherwise noted, EPR measurements represent the average of 10 successive scans spanning 20.48 seconds each. Spectra was fitted to known adducts using Bruker's SpinFit program, which uses characteristic hyperfine coupling constants: superoxide adduct, DMPO-OOH (a_{N}=14.2 G, a_{H}^{β}=11.4 G, a_{H}^{β}=1.2 G); hydroxyl radical adduct, DMPO-OH (a_{N}=14.90 G, a_{H}^{β}=14.93 G). This fitted spectra enabled characterization and quantification of radical species.

### Optical Measurements

Absorption spectrum of the QDs were determined using a UV1600 PC UV-VIS spectrometer (VWR), and the photoluminescence (PL) spectra were taken with a QM-6 steady-state fluorimeter.

### Electrochemical Measurements

Differential pulse voltammetry (DPV) measurements were used to determine the conduction band (CB) and valence band (VB) values of each nanocrystal synthesized. A three-electrode configuration was used with a 2 mm platinum plate electrode, platinum wire, and silver wire as the working, counter, and (quasi-) reference electrodes, respectively. Ferrocene was used as an internal reference. The QDs were suspended in distilled CH₂Cl₂ with 100 mM n-Bu₄NPF₆ as the electrolyte. The whole system was purged with argon for 15 minutes prior to measuring the DPS using a Bio-logic SP200 potentiostat with the following parameters: 50 ms pulse width, 50 mV pulse height, 200 ms step width and 4 mV step height (corresponding to a 20 mV/s scan rate). The CB and VB positions can be determined form the backward (cathodic) and forward (anodic) scan, respectively.

### Bacterial Cell Culture

Colonies of E. coli MG1655 were grown on solid Lysogeny broth/agar plates (LB; Sigma) medium (2% LB; 1.5% agar; Fisher). Individual colonies were picked from and added to 1 mL of 2% LB broth for 16 hours of incubation with shaking (37° C.; 225 rpm). Resulting overnight cultures were diluted 10,000 or 100,000-fold in 2% LB for experiments in 96-well culture plates (Greiner). QD suspensions were sterilized, purified, and washed as described elsewhere herein. InP QDs were then redispersed in LB at known concentration and added to respective wells as a treatment. Bacterial growth was monitored at 30 minute intervals using a Tecan GENios microplate reader, which measured optical density at 590 nm. An ultrathin LED sheet (1.6 mW/cm²) was taped to the ceiling of the microplate reader to provide a constant illumination throughout the experimentation. No abnormal temperature fluctuations were observed, as the temperature was actively held fixed by the instrument at 37° C. Multidrug-resistant clinical isolate carbapenem-resistant E. coli was obtained from the University of Colorado Anschutz Medical Campus. Cell cultures of CRE E. coli were prepared in the same manner as described above, but with cation-adjusted Mueller Hinton broth (CAMHB; VWR), rather than LB. Data presented represents the average of three biological replicates and error bars span two standard deviations.

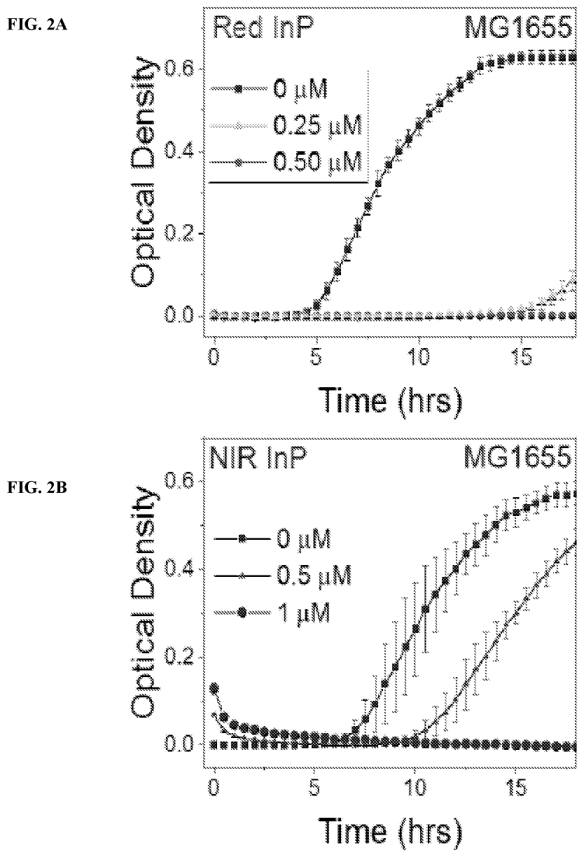

Normalized inhibition values were calculated according to the Equations 2 and 3. The change in bacterial optical density (ΔOD) is measured with respect to OD in the absence of treatment. The timepoint of t=15 h was selected as it represents a saturation of E. coli growth according to the growth curves (FIG. 2), which serves as a conservative and consistent basis across experimental trials.

$$\begin{matrix}Δ {OD}_{t r e a t m e n t} = {OD}_{t r e a t ment , t} - 0 {D}_{no treatnent , t} & Equation 2 \\ Inhibition = \frac{Δ {OD}_{t r e a t m e n t}}{{OD}_{c o n t r o l}} & Equation 3\end{matrix}$$

### Mammalian Cell Culture

Cells were cultured in full growth media containing Dulbecco's Modified Eagle Medium (DMEM, Fisher Scientific) with 10% Fetal bovine serum (FBS, Advanced, Atlanta Biologics), and 50 units/mL of Penicillin-Streptomycin (P/S; Fisher Scientific). For extended durations, HeLa cells were maintained in 10% dimethyl sulfoxide (DMSO), 90% full DMEM freezer stocks. HeLa cells were initialized from freezer stock at passage 3 and subsequently split into three biological replicates which were continuously passaged in parallel. Experiments were performed at passage 6 through 16. Cells were grown at 37° C., 5% CO₂, with humidity control, until 80% confluency. Once cells were about 80% confluent, cells were passaged using 0.25% trypsin (HyClone), counted, and seeded on to 96-well tissue culture treated plates (Fisher Scientific) at a density of 4,500 cells/well in 100 μL. Cell seeding was performed 24 hours prior to treatment (QD addition). After 24 hours of incubation/adherence, control and treatment conditions were prepared in full growth media. Quantum dots were filtered and purified, as described elsewhere herein, and redispersed in full growth media at known concentration. Negative control conditions received only the full growth media, with no QDs. Positive control conditions received only full media (no QDs) for the experiment and were treated with surfactant upon completion, final concentration of 1% Triton X-100 (Sigma) in the well. The 96-well tissue culture plate was then returned to the incubator and illuminated for 18 hours.

A 10× resazurin stock concentration was made by dissolving resazurin in Dulbecco's phosphate-buffered saline (DPBS) to 440 μM. After 18 hours of illumination media was replaced with growth media and 10% resazurin stock, incubated in growth conditions for 4 hours in the dark, and fluorescence measured at 550 nm excitation and 590 nm emission. The surfactant treatment (positive control) is intended to provide 100% toxicity and represent an upper bound for the toxicity assay. Without viable cells, the positive control produces a sort of baseline signal for a resazurin assay. The resazurin turns pink and highly red fluorescent when reduced by metabolic activity (viability).

### Optical Model of Tissue Penetration

In order to estimate the potential for these InP QDs to clear an internal infection, an optical model for light penetration through various tissue was developed. The model considers the optical requirements for eliminating 99.5%+ of bacteria without harming human cells from in vitro experiments: 1) QD dosage (C_{MIC}), 2) light intensity, 3) path length in 96-well plates. This data, when combined using a variation of Beer Lambert law (Equation 4), sums to a value (a) analogous to required dosage of transmitted light for therapy.

$$α = {\sum }_{400}^{700} I n t e n s i {y}_{vitro , λ} \times {C}_{MIC} \times {ɛ}_{QD , λ} \times length \qquad Equation 4$$

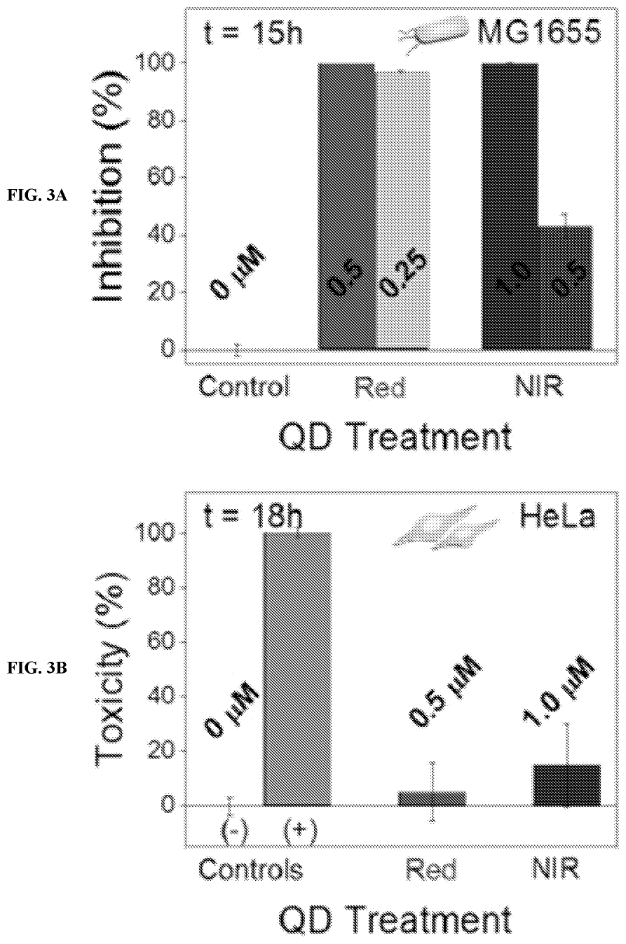

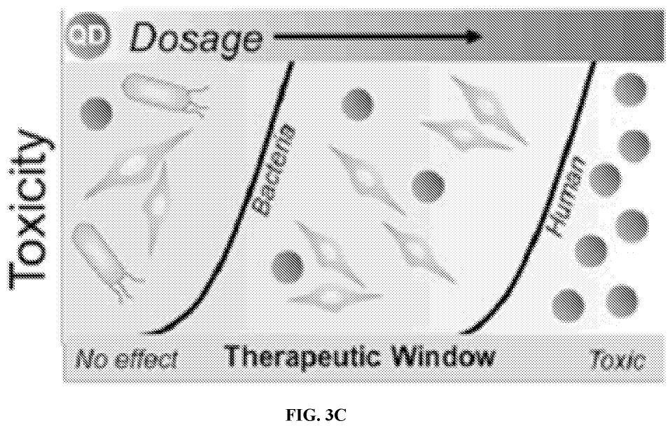

Next, the model considers the following conditions: 1) 2-fold increase in QD concentration (nontoxic due to therapeutic window, as shown in FIGS. 3A-3C, 2) a monochromatic light source of light fluence rate (120 mW/cm²) at a specified wavelength, 3) absorption coefficients for tissues of interest. This data, when combined using Equation 5, allows the model to solve for a path length (z) which satisfies the requirements by setting α=β.

$$β = {Intensity}_{v i v o , λ} \times 1 {0}^{- {ɛ}_{tissue , λ}} \times {C}_{M I C} \times {ɛ}_{Q D , λ} \times z \qquad Equation 5$$

QD optical properties are described elsewhere herein, and tissue absorption coefficients found in the literature, are also described herein and reported in Table 1. Skin: Absorption was divided into epidermis and dermis, based on in vivo coefficients. When solving for path length, epidermis thickness was fixed at 0.007 cm, and dermis thickness was varied.

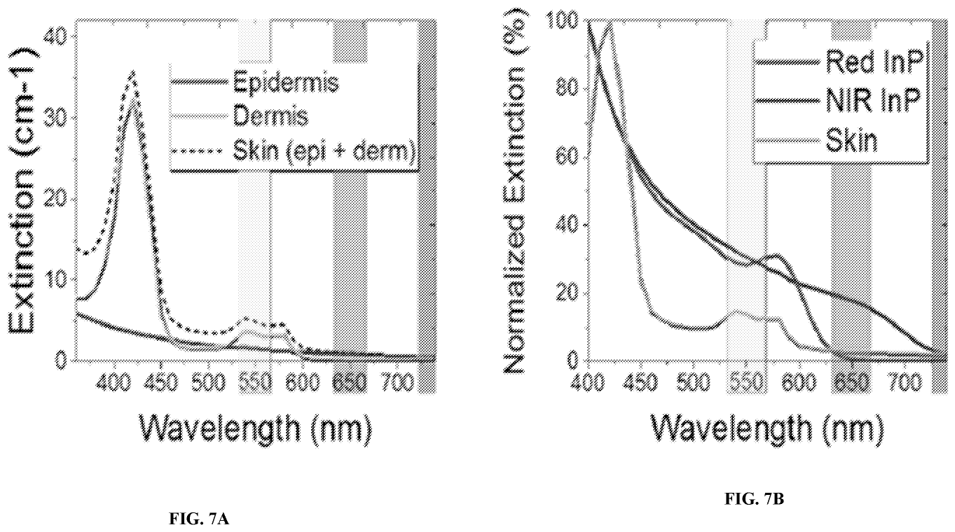

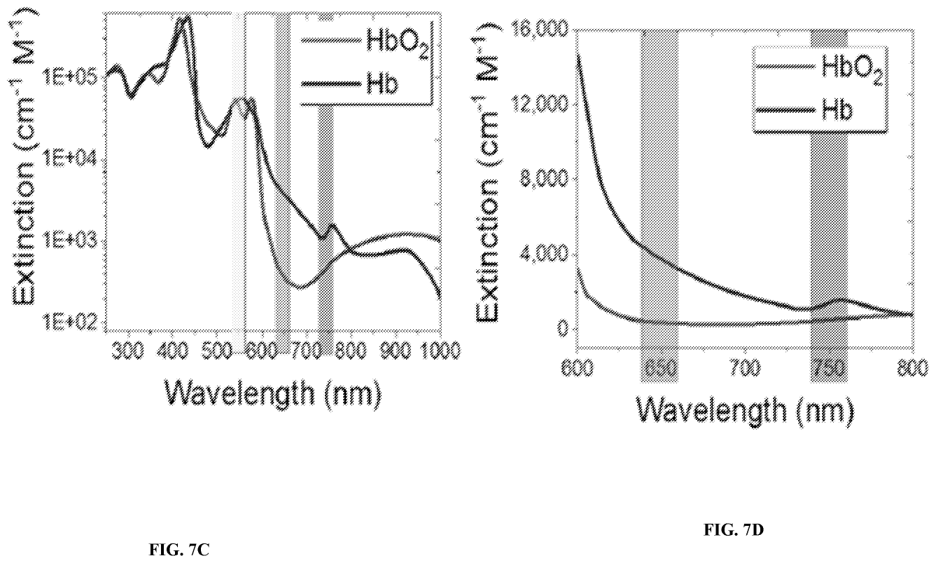

Breast tissue: Data were obtained from a comprehensive review by Sandell, et al. (Sandell, J. L., et al, 2011, Journal of biophotonics, 4(11-12), 773-787). Absorption coefficients at the specific wavelengths of interest (650 and 740 nm) were not included, but were given for 660 nm and 760 nm. Because data were given as ranges, the upper-bound was used in all cases to provide a more conservative approximation of extinction.
Bone: Data were obtained from a clinical study of heel bone by Pifferi, et al (Pifferi, A. et al., 2004, Journal of biomedical optics, 9(3), 474-48). These values appropriately include water, lipid, and blood components as part of the bone.
Lung: Data were obtained from experimental data. (Ntziachristos V. et al., 2002, Opt. Lett. 27, 333-335; Jacques, S. L., 2013, Phys. Med. Biol. 58, R37-61; Beek J. F., 1997, Phys. Med. Biol. 42, 2263-2272). Values assume deflated lungs, which provides a conservative estimate in terms of depth. In the absence of a value at 650 nm, a coefficient corresponding to 630 nm was used, which is also conservative considering the decrease in tissue absorption with increased wavelength. For the absorption at 740 nm, the haemoglobin absorption per known absorption spectra was scaled up (FIG. 7). These values are in good agreement relative to other tissues and are verified values.

TABLE 1
Absorption coefficients used in optical model

| | Epidermis | Dermis | Breast | Bone | Lung |
|---|---|---|---|---|---|
| 550 nm | 1.5 | 3.38 | | | |
| 650 nm | 0.9 | 0.13 | 0.037-0.110 | 0.115 | 0.5-0.96 |
| 740 nm | 0.6 | 0.06 | 0.031-0.10 | 0.075 | 0.155 |

Zn₁₋ₓCdₓTe Quantum Dots

### CdTe-2.4 QD Synthesis.

QD synthesis was carried out via hydrothermal method. The tellurium precursor was prepared by dissolving 33 mg of sodium borohydride (Fisher) in 1 mL of degassed water for injection into a 2 mL vial containing 40 mg of tellurium powder (Alfa Aesar). The Te-precursor was left to react in a fume hood for 90 min until a magenta colored solution (NaHTe) was obtained. 6.48 mg of zinc nitrate hexahydrate (21.8 mmol; Sigma) was added to 10 mL of degassed water containing 1.8 μL of 3-mercaptopropionic acid (MPA; Alfa Aesar) to serve as a cationic precursor. The reaction mixture (750 μL of Zn²⁺ precursor; 750 μL of degassed water; 2.5 μL of Te precursor) was adjusted to pH 11 and allowed to react at 98° C. for 60 min until a colorless ZnTe QD suspension was obtained. For Zn₁₋ₓCdₓTe QDs, the cationic precursor solutions varied in their Zn²⁺:Cd²⁺ molar ratios (1:1, 5:1, 10:1, 20:1). A greater volume (10 μL) of Te precursor was used in the reaction mixture in order to favor ZnTe bond formation. Zn₁₋ₓCdₓTe QDs reaction proceeded for 30 min at 98° C. CdTe-2.4, ZnTe, and Zn₁₋ₓCdₓTe QDs were washed 3 times with pH 11 water using Nanosep 3k filters (Pall) and centrifugation at 10000 rpm for 7 min per cycle. Poorly passivated QDs were separated by centrifugation (4000 rpm). Sterile techniques were maintained during all QD synthesis.

### QD Characterization.

QD UV-vis absorbance spectra were measured using a VWR UV-1600PC UV/vis spectrophotometer. Emission spectra were obtained on a calibrated PTI fluorimeter. QD composition was determined using inductively coupled plasma mass spectrometry (ICP-MS). Composition data represent the average of three replicates, and error bars span two standard deviations from the average.

### Electrochemical Measurements.

Differential pulse voltammetry (DPV) was used with a Bio-logic SP-200 potentiostat/galvanostat to determine Zn₁₋ₓCdₓTe CB, VB positions. Using a three-electrode (working electrode of glassy carbon; counter electrode of platinum wire; reference electrode of Ag/AgCl) configuration, the experiments were conducted using a scanning rate of 20 mV/s (parameters: 50 ms pulse width, 50 mV pulse height, 200 ms step width, and 4 mV step height). The forward scan range was from −0.5 V to 1.5 V. The backward scan range was from 0.5 V to −1.8 V. Deionized water purged with argon (30 min) was used as a solvent, and sodium sulfate (Fisher) as the electrolyte. All the experiments were carried out at room temperature.

### Superoxide Detection.

Superoxide generation was confirmed using a Bruker Elexsys E 500 electron paramagnetic resonance (EPR) spectrometer with an SHQE resonator operated in a dark room. A power of 5 W and a microwave attenuation of 16 dB was used. 100 μL aliquots of QD suspensions were prepared for EPR by adding 1 μL of spin-trapping agent 5,5-dimethyl-1-pyrroline N-oxide (DMPO) in dark conditions. DMPO forms stable detectable adducts with radical species such as superoxide. After loading sample suspension into quartz capillaries, a baseline EPR spectrum was measured in the dark. Then, the sample was exposed to 45 s of a visible light source and quickly returned to the EPR for measurement. Excitation for ZnTe involved ultraviolet light (312 nm). All EPR measurements represent the average of 10 successive scans (20.48 s each). Radicals were identified and quantified using Bruker's SpinFit program and characteristic hyperfine coupling constants for the superoxide adduct DMPO-OOH (a_{N}=14.2 G, a_{H}^{<}=11.4 G, a_{H}^{β}=1.2 G) and the hydroxyl radical adduct DMPO-OH (a_{N}=14.90 G, a_{H}^{β}=14.93 G).

### Bacterial Cell Culture and Therapeutic Analysis.

Individual colonies of E. coli MG1655 were picked from solid Luria-Bertani (LB; Sigma) medium (2% LB; 1.5% agar; Fisher) and added to 1 mL of LB broth (2%) for overnight incubation (16 h; 37° C.; 225 rpm shaking). Resulting cultures were diluted 1:100 in fresh LB medium (2%) for experiments in culture plates (96-well plates; Greiner). CdTe-2.4, ZnTe, Zn₁₋ₓCdₓTe suspensions were washed and redispersed in LB and added to respective wells as a treatment. Bacterial growth was monitored via optical density (590 nm; 30 min intervals) using a microplate reader (Tecan GENios). A thin LED sheet (1.6 mW/cm², FIG. 26) was taped to the ceiling of the microplate reader to provide a constant light source. The temperature was held fixed at 37° C. Clinical isolates (carbapenem-resistant E. coli) were obtained from the University of Colorado Anschutz campus. CRE E. coli cell cultures were conducted in the same manner as described above with the exception of the medium used: cation-adjusted Mueller-Hinton broth (CAMHB; VWR). Data represent the average of three biological replicates, and error bars span two standard deviations.

### Mammalian Cell Culture.

HeLa cells, maintained long-term in 10% dimethyl sulfoxide (DMSO) freezer stocks, were cultured in Dulbecco's modified Eagle medium (DMEM, Fisher Scientific), 10% fetal bovine serum (FBS, Advanced, Atlanta Biologics), and 50 units/mL penicillin-streptomycin (P/S; Fisher Scientific). HeLa cells were started from a freezer stock at passage and split into three separate biological replicates which were continuously passaged as separate biological replicates; experiments were performed at passage 6. HepG2-C3A hepatic cells and MC3T3-E1 osteoblasts were also used for toxicity experiments. These cell lines were cultured using the same protocol as HeLa cells, with minor differences: MC3T3 cells were cultured using minimum essential media-α (MEMα, Fisher Scientific) rather than DMEM, as this is the standard method (HepG2 were grown in DMEM); MC3T3 and HepG2 were seeded at passage 10. Cells were grown at 37° C., 5% CO₂, and controlled humidity to 80% confluency and passaged using 0.25% trypsin (HyClone) onto 96-well tissue culture treated plates (Fisher Scientific) at a density of 4500 cells/well 24 h prior to QD addition. After 24 h of incubation on tissue-culture treated plates, treatment and control conditions were prepared in DMEM/FBS/P/S media. Quantum dots were filtered and purified, as described above, then mixed with media to the appropriate treatment dose. Negative control conditions received only the full media. Positive control conditions received full media for the experiment and upon completion of the experiment were treated with 10% Triton X-100 (Sigma) for a final concentration of 1%. The 96-well tissue culture plate was then returned to the incubator and illuminated with a visible LED sheet (1.6 mW/cm²) for 18 h.

### Lactate Dehydrogenase Assay.

Following 18 h of incubation with QDs, 50 μL of water was added to each well to achieve 150 μL of supernatant, and 90 μL of this supernatant was used to measure lactate dehydrogenase (LDH) release by cells with the CytoSelect LDH cytotoxicity assay kit. LDH release correlates to cell toxicity, and percent toxicity can be measured using the following equation:

$$percent toxicity = \frac{( {Abs}_{450 nm}^{treatment} - {Abs}_{450 nm}^{negative control} )}{( {Abs}_{450 nm}^{positive control} - {Abs}_{450 nm}^{negative control} )} \qquad Equation 6$$

### Growth Calculations.

Normalized inhibition was calculated according to Equations 7 and 8. The change in bacterial optical density (ΔOD) is measured with respect to OD in the absence of treatment.

$$\begin{matrix}Δ {OD}_{treatment} = {OD}_{treatment , t} - {OD}_{no treatment , t} & Equation 7 \\ inhibition = \frac{Δ {OD}_{treatment}}{{OD}_{control}} & Equation 8\end{matrix}$$

### GIC₅₀ Method

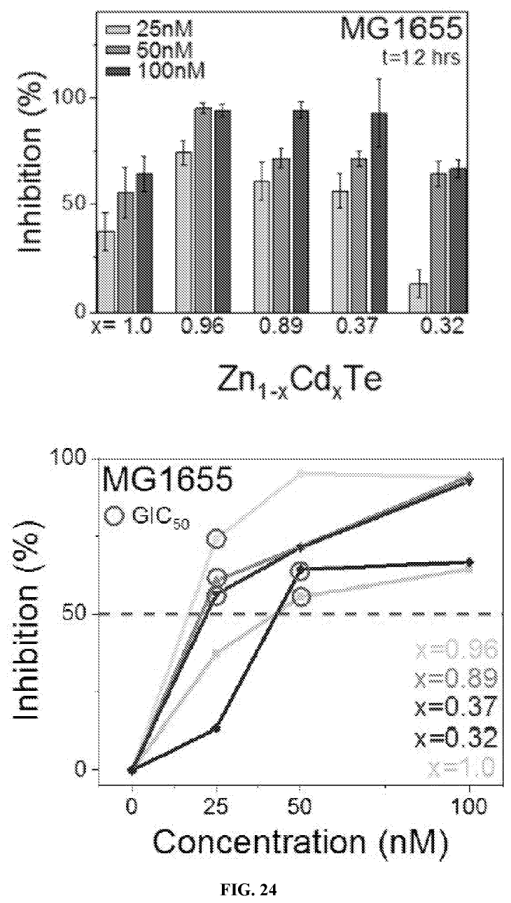

To determine GIC₅₀ values for use in subsequent calculations, phototoxicity data obtained from bacterial cell culture in the presence of QDs and light was used. GIC₅₀ values were calculated individually for each QD of varying composition (x). First, inhibition at t=12 h was calculated using Equation 8. t=12 h was selected as time-point, as this time-point showed equilibrated growth in our no-treatment control. Because discrete values of QD dosage (12.5, 25, 50, 100 nM) were tested, each GIC₅₀ was selected as the minimum dosage that demonstrated at least 50% inhibition (FIG. 24).

### Effective Cadmium Content for Nanotherapy

Effective cadmium content for therapy using these QDs was determined by analyzing the peak phototherapeutic effect versus cadmium content. Data from ICP-MS yielded amounts of cadmium per QD suspension with units of (ppb of Cd²⁺/nM QD). Next, cell culture data provided the concentrations needed for each different QD to inhibit bacterial growth by 50% (GIC₅₀ values) in units of nM. The product of these two values is a metric (with units in ppb) that corresponds to cadmium content for therapeutic effect. The QD with the lowest value was deemed to be optimal, as this corresponds to a minimum of cadmium content. ZnTe was not included in this analysis because it contained no cadmium and presented no therapeutic effects.

### Example 1: EPR Analysis

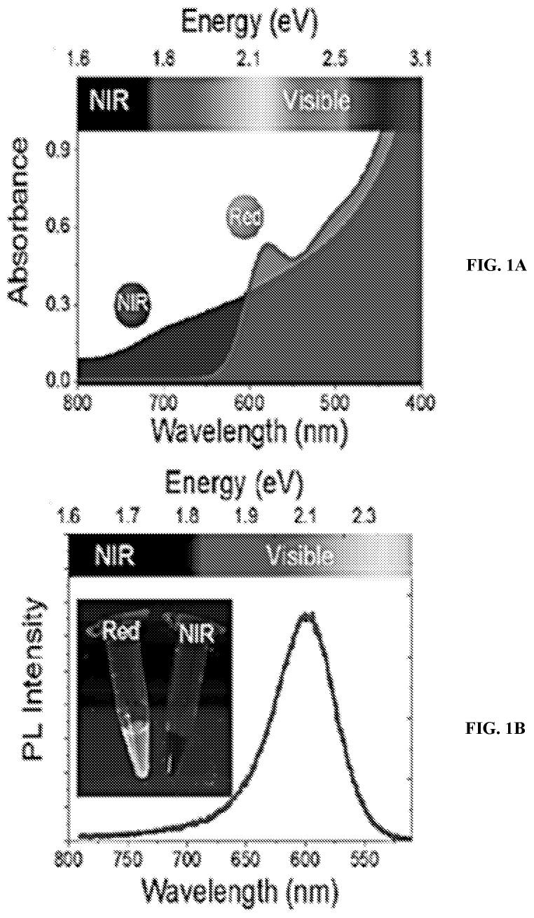

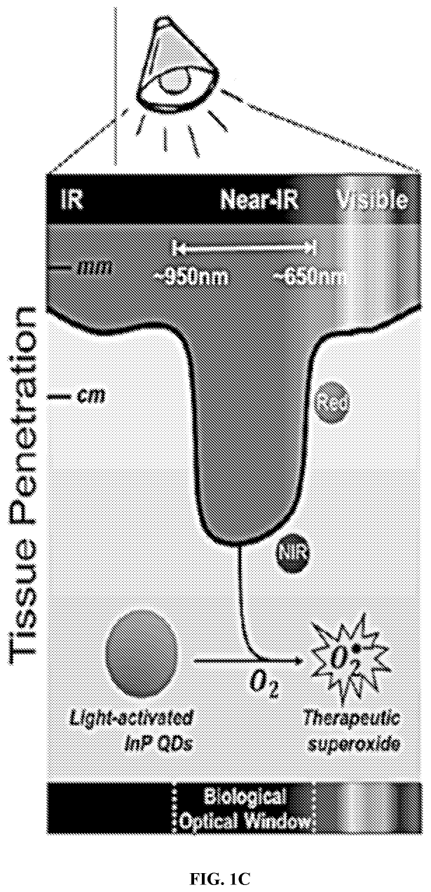

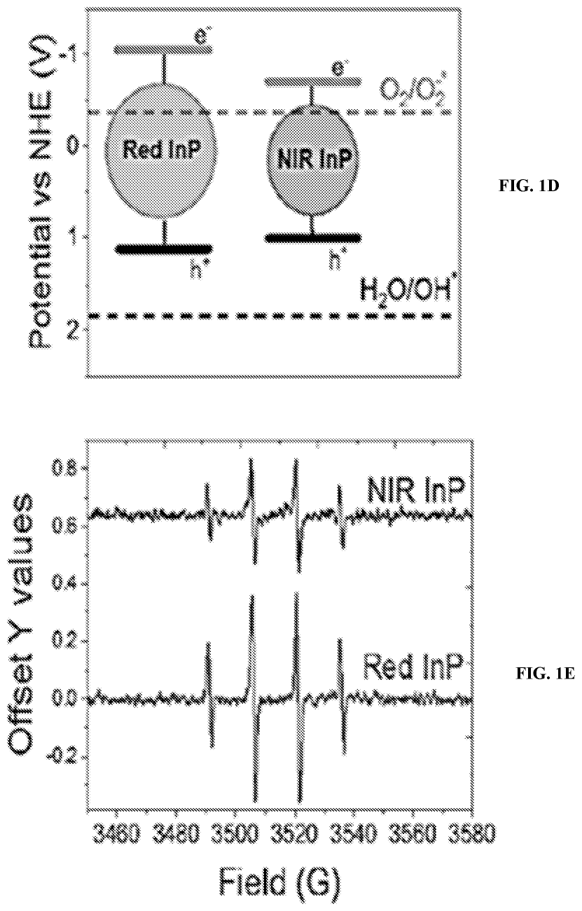

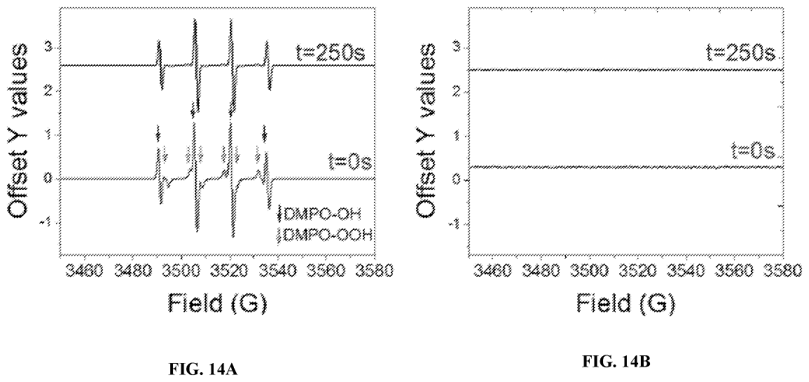

In using EPR spectroscopy, it was found that both InP QDs, upon illumination, generate strong radical signals (FIG. 1E). It is worth noting that major peaks shown correspond to DMPO-OH—the spin-trap adduct of hydroxyl radicals with 5,5-dimethylpyrroline-N-oxide (DMPO). This more stable adduct forms as a result of the decomposition of DMPO-OOH, the superoxide radical adduct. This effect can also be tracked by observing how the EPR spectra changes with time (FIG. 14).

### Example 2: InP QD Growth Rate and Lag Effects

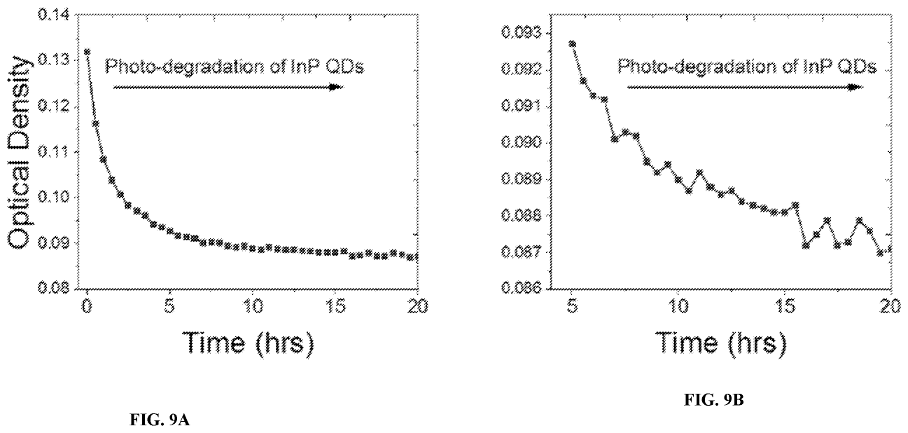

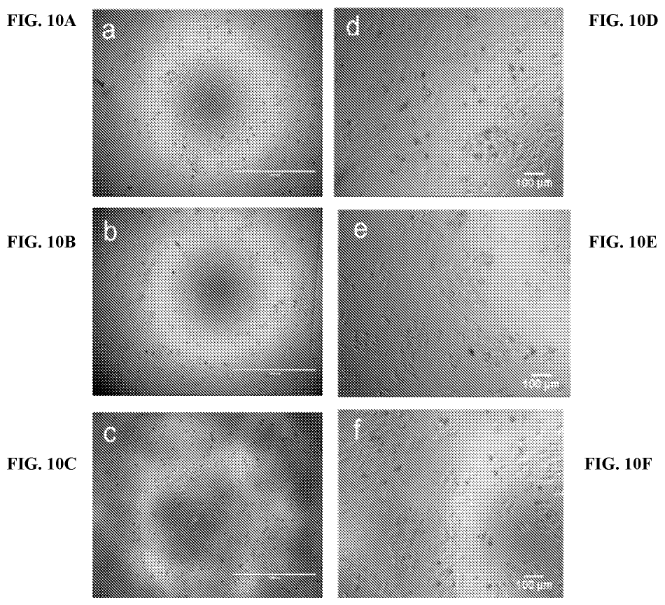

Both InP QDs demonstrated clear growth inhibition of bacteria after incubation in presence of light. Also, worth noting is the apparent decrease in optical density observed in NIR InP treatment, relative to absorbance at t=0 h, due to QD oxidation or photo-bleaching. Since NIR InP absorbs more light than Red InP, its optical density in a 96-well plate was higher at similar concentrations (FIGS. 2A-2B), and its presence is even apparent to the naked eye. However, that dark color does not persist over the full duration of the cell assay. It was observed that prolonged exposure to light causes gradual photo-bleaching for approximately 10-15 hours (FIG. 9). This apparent difference between Red and NIR InP does not imply that Red InP are immune to photo-oxidation. Rather, the same process of photo-degradation is occurring in the Red InP, albeit less visibly, and affecting the therapeutic effect.

Following generation of photoexcited electron-hole pairs in these QDs, there are several available pathways for them. The electron-hole pair can recombine to provide emission/PL, can be transported to the surface and injected to matched/available chemical species like oxygen to form superoxide (photochemistry), or combine non-radiatively through a number of photophysical phenomenon such as Auger recombination, defect-mediated recombination, surface-mediated recombination and other pathways. While PL emission and photochemistry (radical formation) are competing pathways, the majority of charge carriers are recombined non-radiatively in QDs (>80-90%), and hence sometimes PL emission can be used as a measure of surface passivation or photo charges available for radiative emission or photochemistry.

In addition to available electron-hole pairs, as with PL emission, QD photochemistry or radical generation additionally requires: 1) efficient transport of photogenerated charges to the QD surface/adsorbate site (oxygen here); 2) availability of available adsorbates/chemical species on the QD surface; and 3) rapid charge injection from the QD core (InP here) to the adsorbed chemical species (oxygen here). Using the high reduction potential of designed InP conduction band (measured redox potentials relative to oxygen shown in FIG. 1D) and resulting available overpotential, the charge injection is also found to be rapid. Therefore, like PL, the superoxide generation is also primarily limited in freshly prepared QDs simply by surface defects and non-radiative recombination pathways. Hence the PL emission spectra is presented as a characterization of the QDs surfaces.

Additionally, with increasing surface photo-oxidation, the growing surface oxide layers produces a thicker tunneling barrier and hence interfacial resistance to charge injection to adsorbed oxygen species, resulting in slower charge injection and lower superoxide generation with time, effectively decreasing the therapeutic efficiency and making the QDs ineffective after ˜15 hours.

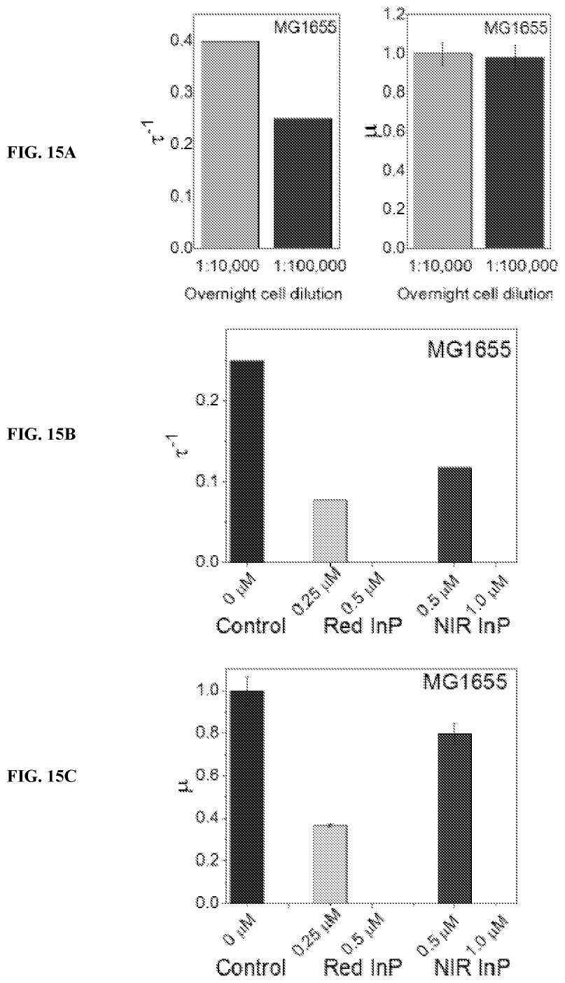

The growth kinetics of MG1655 treated with different doses of Red and NIR InP were evaluated (FIGS. 15A-15C). Two metrics were used to quantify the length of lag phase and the exponential growth rate, r and p, respectively. At low doses of Red InP and NIR InP (250 nM and 500 nM, respectively), a significant delay in growth was observed. At minimum inhibitory concentrations (MIC; 500 nM and 1000 nM, respectively), no growth was observed. For the lower doses, bacterial growth rates in late time points remain much lower in treated versus control conditions-approximately 20 to 60% slower. Such slowed growth suggests a reduced flux of QD-generated therapeutic superoxide. Taken together, these findings, in some embodiments, indicate a 10-15 hour lifespan of InP QD treatment.

### Example 3: Optical Model of Tissue Penetration

Optical model described here was used to estimate the depth to which monochromatic red/near-IR light could penetrate tissue and activate therapeutic InP QDs. One proposed application was for killing pathogens localized in the lungs, such as Mycobacterium tuberculosis and Pseudomonas aeruginosa. As such, the chest/lung model was selected as appropriate for this scope. One could imagine light coming through the chest or the back to reach QDs in the lung to treat an infection there, so optical properties of skin, breast, bone, and lung were required for the analysis. Because the QDs are sensitive to light at 650 nm and 740 nm (NIR InP), these monochromatic wavelengths were selected for analysis, along with a lower wavelength reference point (550 nm).

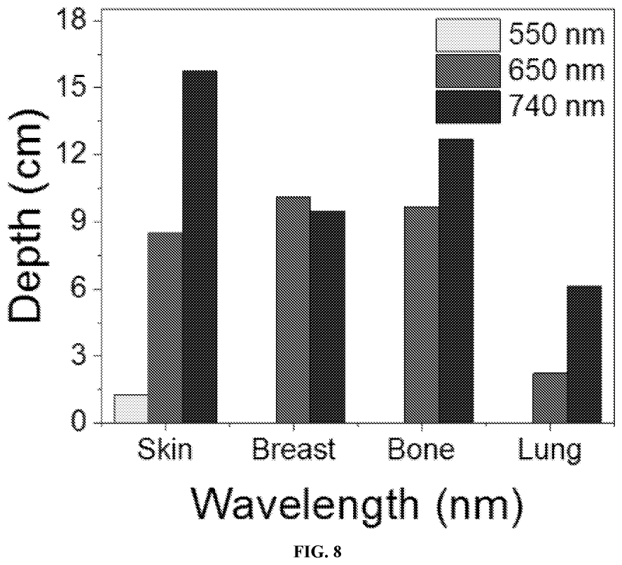

In solving Equations 4 and 5, it was confirmed that higher wavelengths of light do indeed penetrate deeper. Remarkably, QDs can be sufficiently activated from light that travels through several centimeters of skin-up to approximately 16 cm of skin-far more than is required (FIG. 8). This means that enough light can penetrate the epidermis and dermis to reach underlying tissue layers. Considering the optical properties of breast tissue, it was once again found that sufficient light can penetrate 8-9 cm of the tissue for therapeutic activation. The analysis reveals the same for bone (slightly deeper for 740 nm), and lung tissue. Less penetration is observed for the lung tissue (2-6 cm), but values used are conservative (see methods), and other metrics can be tuned. One of the tunable metrics is light intensity. The evidence of deep light penetration described herein suggests that this therapeutic is suitable for in vivo application.

### Example 4: Design, Application, and Therapeutic Effectiveness of Red and NIR Indium Phosphide (InP) QDs

Using conventional, scalable methods, InP QDs of varying size and, therefore, tunable optoelectronic and resulting redox properties were synthesized (FIGS. 1A-1E). Both “Red InP” and “NIR InP” show strong photo-electrochemical response to low-energy NIR light, as well as strong light-induced cell-killing of Escherichia coli, and an MDR clinical isolate prioritized as a “critical” pathogen by the WHO. At the low doses required for therapeutic antimicrobial action against the pathogen, is shown that both InP QDs are nontoxic to host human cells.

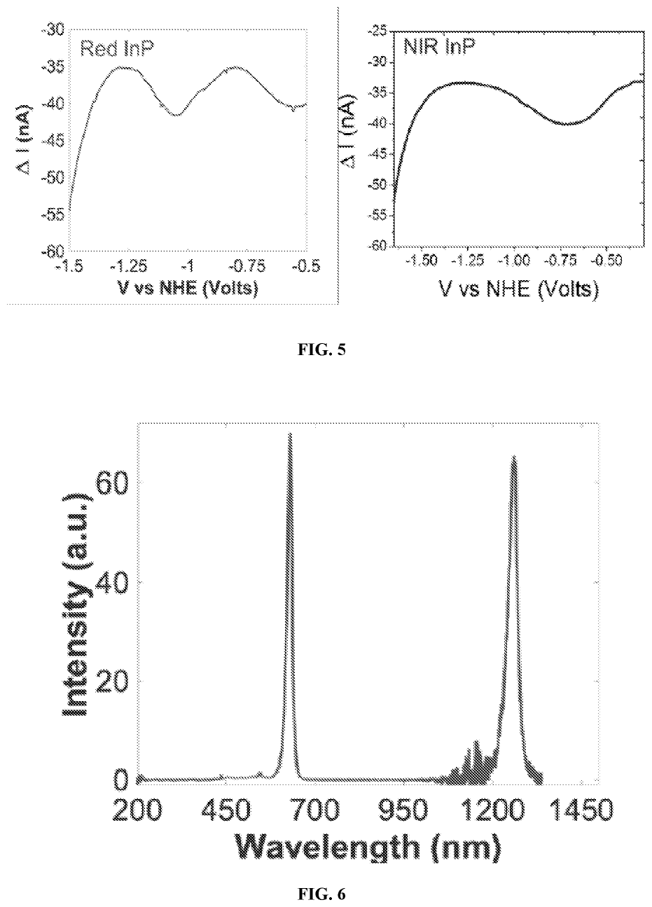

InP was chosen as a potential nanotherapeutic material since it has a narrow bulk band gap of 1.35 eV, contains no heavy metals, and is associated with few mammalian cell toxicity concerns. InP QD synthesis was carried out in organic phase and, to enhance chemical stability and biocompatibility, a thin shell (ZnS or ZnSe) was added to the InP surface. Both QDs are small, and water-soluble, as they are decorated with the negatively-charged ligand 3-mercaptopropionic acid (MPA). The “Red InP” shows strong absorbance into red wavelengths >650 nm, with an excitonic peak around 575 nm and emission peak centered around 601 nm; the “NIR InP” absorbs light well into the NIR (>700 nm), with an excitonic peak around 720 nm (FIGS. 1A-1B). These optoelectronic properties align well with the biological optical window (FIG. 1C). Using differential pulse voltammetry (DPV), these QDs were measured to have conduction band (CB) positions at −1.052 and −0.71 eV vs. NHE at pH 7, respectively, well above the threshold for superoxide generation (−0.33 eV) (FIGS. 1D, 5). Such optoelectronic and electrochemical properties suggest that these QDs could generate superoxide when illuminated with low-energy red light (FIG. 6). Indeed, using electron paramagnetic resonance (EPR) spectroscopy, it was found that both QDs generate a strong radical signal in the presence of light (FIG. 1E). Taken together, these results confirm the promise of red- and NIR-absorbing, superoxide-generating InP QDs. Below 650 nm wavelengths, human tissue absorbs light (FIG. 7). Between 650 and 950 nm, however, light-penetration exceeds several centimeters. While light scattering in biological tissues is a concern for NIR QD bio-imaging, it is less of a concern for therapeutic QDs. Here, light could be administered at very high flux using NIR LEDs, would only need to travel into the tissue, and could come from all directions. Pathogens localizing in the lung, such as Mycobacterium tuberculosis and Pseudomonas aeruginosa, would be well within the reach of NIR-nanotherapy. Based on an optical model of skin, breast, bone, and lung tissue, monochromatic light (650 nm to 740 nm) can deeply penetrate several layers of tissue to sufficiently activate InP QDs for therapy (FIG. 8). It is also worth noting that M. tuberculosis is known to require high-oxygen environments, and that both pathogens possess advanced iron-sequestration strategies-two characteristics which increase their vulnerability to superoxide therapy In order to test the efficacy of the superoxide-generating QDs, the in vitro growth of a strain of E. coli (MG1655) treated with InP in the presence of light was assessed. In a control (“No Treatment”), MG1655 enters the exponential phase approximately 4 hours into the experiment (t=4 h). When treated with 250 and 500 nM of Red InP, significant cell-killing relative to the control was observed (FIG. 2A). After 20 hours, the 500 nM Red InP condition shows no signs of bacteria proliferation. The 250 nM condition shows similar toxicity until a period of slow growth begins around t=13 h, possibly due to QD oxidation (FIGS. 9, 15). 500 and 1000 nM doses of NIR InP display similarly bactericidal behavior (FIG. 2B). In this case, no bacteria proliferation was observed until after t=8.5 h for the lower dose, and no proliferation for the higher dose. The degree of growth inhibition was quantified at a specific time point, t=15 h, corresponding to saturated growth in no treatment, to evaluate nanotherapeutic efficacy. Relative to no treatment, Red InP and NIR InP QDs inflict greater than 99.5% bacteria-killing (FIG. 3A). It is worth noting some differences between the effects of Red and NIR InP. The bactericidal dosage, or minimum inhibitory concentration (MIC, 500 nM for Red InP and 1000 nM for NIR InP), apparently increases as absorbance shifts to lower energies. This may be a consequence of QD size, shell or surface discrepancies-any of which may affect biomolecule interactions, cellular uptake, stability, or superoxide generation rates.

With evidence for the therapeutic effect of photo-activated InP QDs in bacteria, it was sought to evaluate the toxicity of InP QDs to human cells. HeLa cells were treated with bactericidal doses of Red InP and NIR InP in the presence of light. No significant toxicity was observed at these QD doses using a standard resazurin assay, relative to positive and negative controls (FIG. 3B). Results were confirmed using microscopy, which shows no significant changes to cell density or morphology in the presence of InP QDs (FIGS. 11A-11D). Bactericidal concentrations of both Red InP and NIR InP show no significant toxicity or morphological changes in the HeLa cells after 18 hours of treatment. When these concentrations were increased further—to 1 μM Red InP and 2 μM NIR InP—a slight decrease in HeLa viability was observed and some morphological changes (FIG. 11E). However, these extreme concentrations are two-fold higher than those shown suitable for >99.5% bacterial toxicity, and at least four-fold higher than concentrations sufficient for therapeutic effect. Therefore a “therapeutic window” whereby light-activated InP QDs may kill pathogenic bacteria while leaving host human cells unharmed can be safely deduced (FIG. 3C).

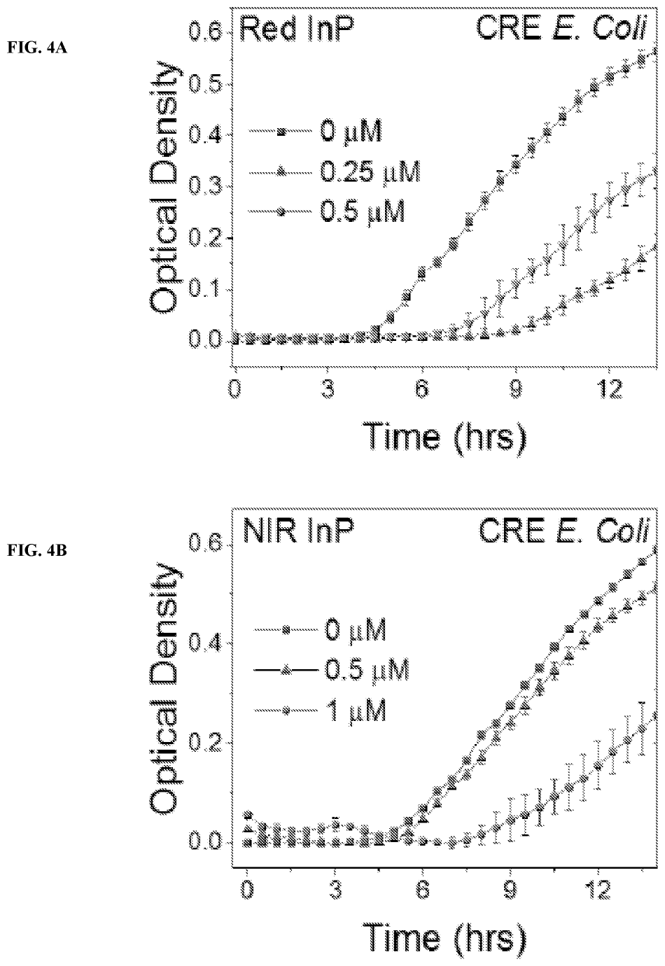

The suitability of Red InP and NIR InP for treating multidrug-resistant infections was evaluated. The strain of carbapenem-resistant Enterobacteriaceae E. coli (CRE E. coli) used herein is classified as the most critical pathogen, according to the WHO, because it is resistant to multiple classes of antibiotics, including carbapenem—a “last resort” antibiotic. Upon treatment with Red InP (FIG. 4A) or NIR InP (FIG. 4B) and light, significant growth inhibition of the critical pathogen was observed, with clear extension of the MDR bacteria lag phase. Although the inhibition at the lowest doses is less pronounced than in the lab-strain, the effective doses are still well below thresholds for human cell toxicity. This therapeutic window of dosage suggests that Red InP and NIR InP QDs are promising nanotherapeutics for treating MDR bacterial infections.

Based on the clear evidence of superoxide generation from the InP QDs, the observed toxicity to bacteria was expected. Bacterial toxicity thresholds are lower for superoxide than for other ROS, and bacteria are more susceptible to superoxide than are human cells. Further, since pathogens sequester iron from their hosts and environment in order to proliferate, they become conveniently iron-rich targets for superoxide therapy. Indeed, designing QDs specifically for this purpose demonstrates the potential for the rational design of a nanotherapeutic: One that is acutely toxic to bacteria, and nontoxic to human cell lines (FIGS. 3A-3C). These biocompatible QDs are free of toxic constituent materials, including heavy metals. The use of a shell prevents oxidation and elemental leaching and makes the QDs more stable. QD size also plays a role in the therapeutic effect, since these inorganic nanoparticles are not metabolized. With diameters below 6 nm (FIGS. 12A-12C), Red InP and NIR InP are small enough for diffusion into bacteria and renal clearance important for intracellular superoxide generation and preventing bioaccumulation in vivo, respectively.

In conclusion, the development of therapeutic nanoparticles (nanotherapeutics) capable of selectively killing MDR bacteria using low-energy Near-IR light as a trigger are presented herein. Both the “Red InP” and “NIR InP” can generate intracellular superoxide radicals using red/NIR light, and appear uniquely appropriate for use as bacterial nanotherapeutics without the limitations seen in other nanomaterials. This behavior makes them strong candidates for phototherapy, which was demonstrated in cell culture with lab strain of E. coli and an MDR Priority I “critical” pathogen. This acute bactericidal effect does not translate to human cell toxicity, according to our findings. At doses deemed therapeutically effective for killing wild-type and MDR bacteria, human cells are left intact. Beyond the apparent biocompatibility, these heavy metal-free QDs can be sensitized by deep tissue-penetrating light. The in vitro results here show great promise that these QDs can serve as an urgently needed antimicrobial alternative, capable of selectively killing the world's most dangerous pathogens.

### Example 5: Design, Application, and Therapeutic Effectiveness of Zn₁₋ₓCdₓTe QDs

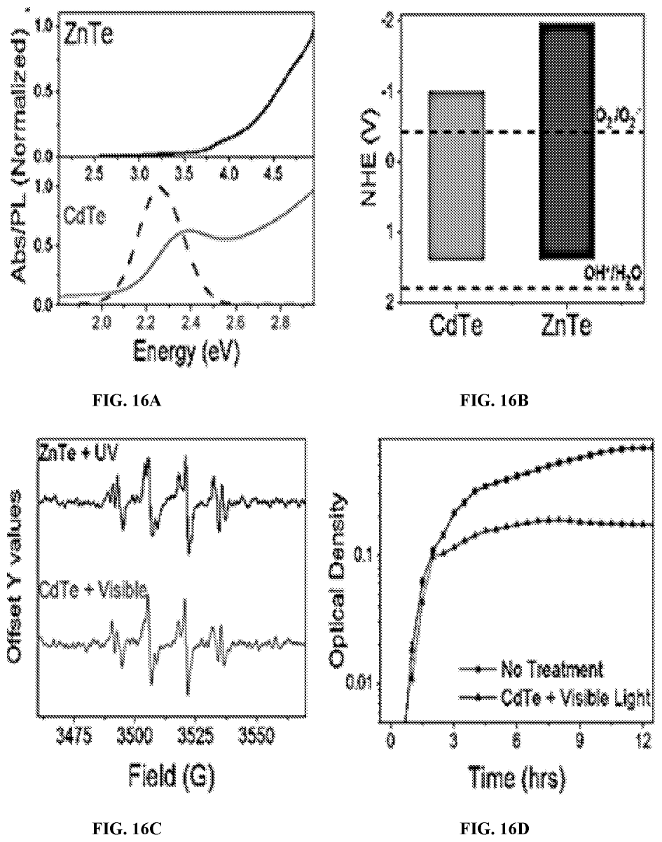

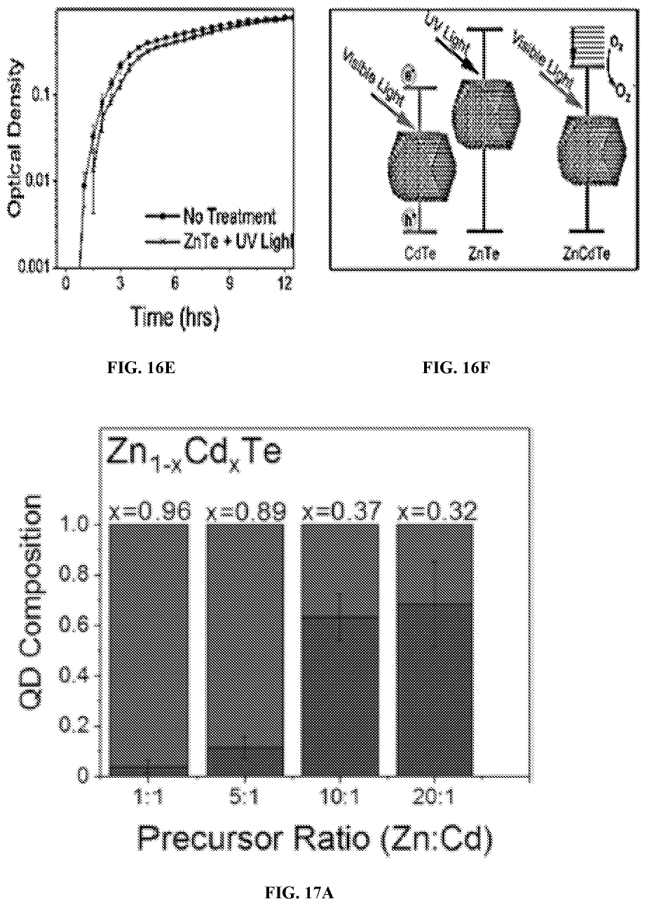

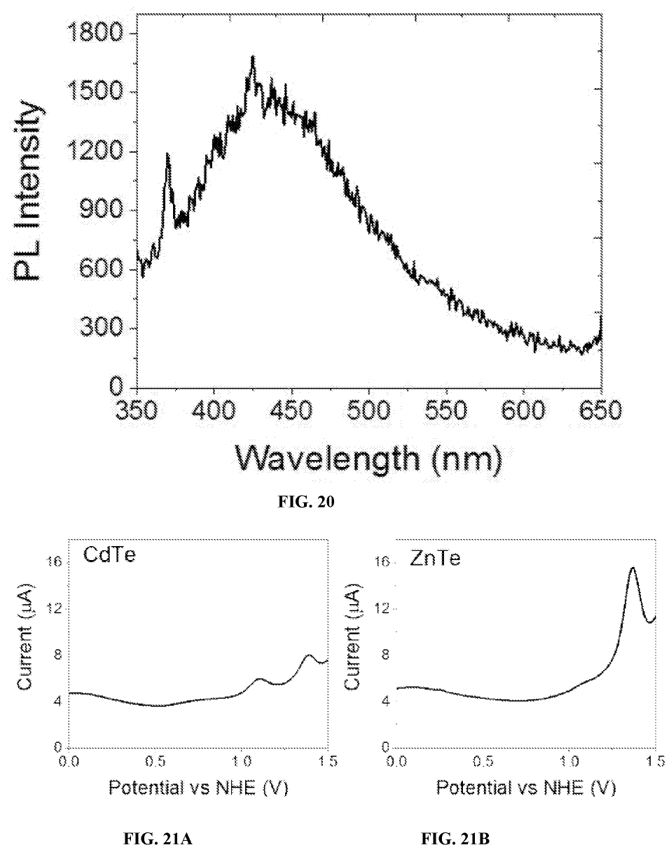

To reduce the cadmium content in a QD-superoxide therapy, firstly zinc telluride QDs were synthesized. Zinc telluride is a semiconductor with a direct bandgap of 2.26 eV in bulk (versus 1.5 eV for CdTe in bulk). Synthesized ZnTe QDs showed a weak excitonic peak at around 330 nm and a broad tail extending to higher wavelengths (FIG. 16A). In contrast, CdTe-2.4 QDs clearly showed visible light absorbance with an excitonic peak near 520 nm. CdTe-2.4 QDs also showed a strong fluorescence peak centered ˜549 nm (FIG. 16A). The ZnTe QDs exhibited negligible fluorescence when measured (FIG. 20). The observed bandgap of synthesized ZnTe QDs was 3.3 eV, versus 2.4 eV for CdTe-2.4 QDs. Due to the heavy-hole states from tellurium, no significant difference in the position of the ZnTe QD VB relative to that of CdTe-2.4 QDs (FIGS. 21A-21B) was observed. With their similar VB position and large bandgap, ZnTe QDs had a higher (more negative) CB position and greater consequent reduction in electrochemical measurements. As observed in our DPV measurements, the ZnTe QD CB shifts up relative to that of CdTe-2.4 QDs to well above the threshold for superoxide generation (FIG. 16B).

In order to validate the ability of ZnTe QDs to generate superoxide radicals, electron paramagnetic resonance (EPR) spectroscopy coupled with spin-trapping was used. Upon illumination with a UV light source, characteristic peaks corresponding to superoxide and hydroxyl radical adducts were detected: DMPO-OOH and DMPO-OH, respectively (FIG. 16C). The peaks detected fit with theoretical hyperfine coupling constants and proportionality factors (DMPO-OH, a_{N}=14.90 G, a_{H}^{β}=14.93 G, g=2.006; DMPO-OOH, a_{N}=14.2 G, a_{H}^{β}=11.4 G, a_{H}^{β}=1.2 G, g=2.006). As known, with CdTe-2.4 and other sources of superoxide, the DMPO-OH adduct was formed indirectly via decomposition of DMPO-OOH and not via direct injection of photo-generated holes from the QDs to water for hydroxyl radical generation. This is supported by the VB position for both CdTe-2.4 and ZnTe QDs which lies below the threshold for OH generation, as well as further electrochemical measurements in the presence and absence of dissolved oxygen. A comparable EPR spectrum was observed for CdTe-2.4 QDs illuminated by visible light (FIG. 16C); ZnTe QDs generated no measurable radical species when illuminated by visible light.

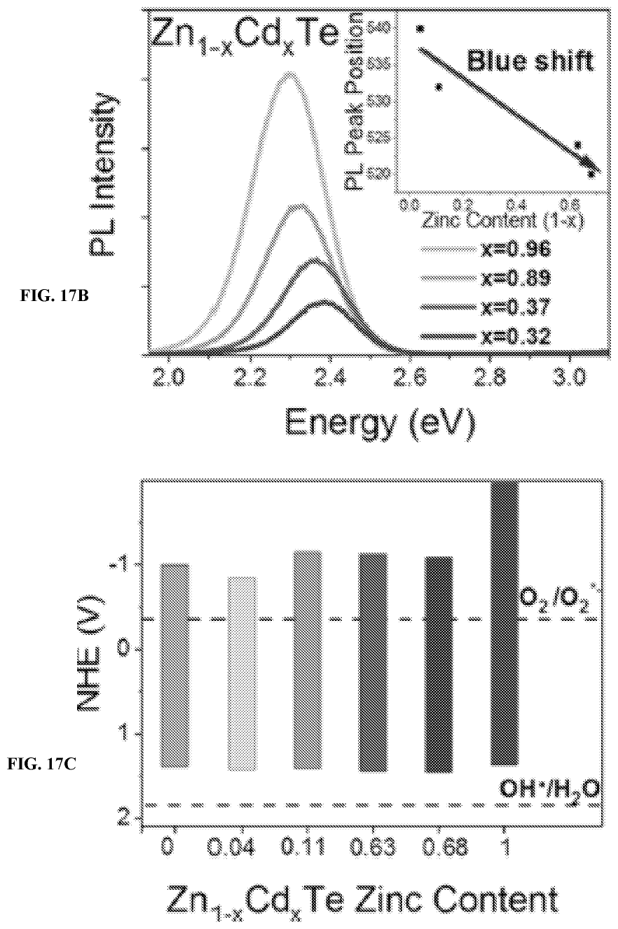

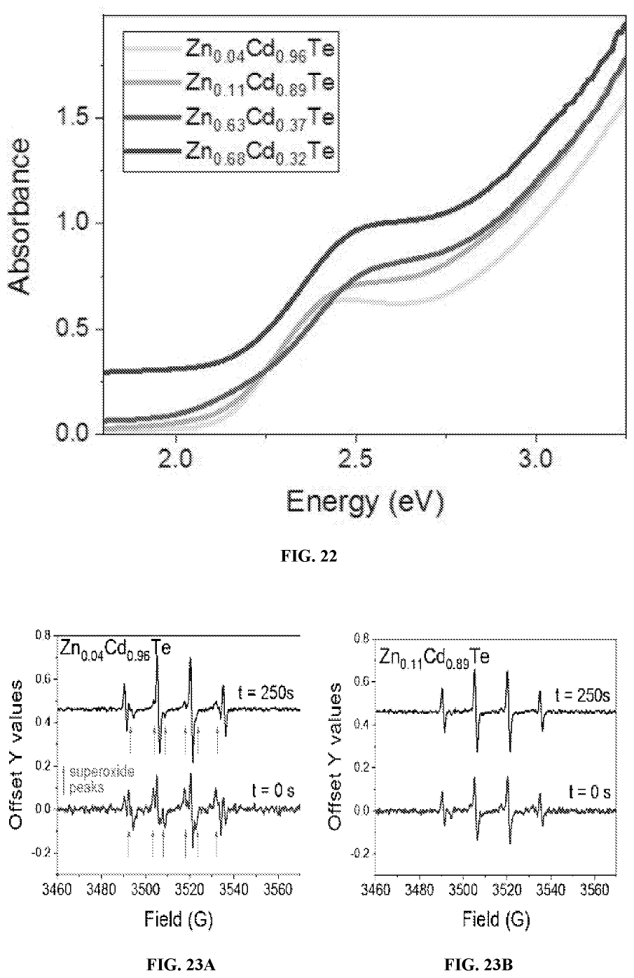

To evaluate the composition-tuned ternary Zn₁₋ₓCdₓTe for intracellular superoxide generation and their therapeutic potential, CdTe-2.4 QDs was utilized as a positive control and monitored the superoxide generation responsible for the selective killing of bacteria in the presence of visible light (FIG. 16C). ZnTe QDs (x=0) could not generate intracellular superoxide with visible light. As such, further tests on bacterial growth in cell culture in the presence of visible light and ZnTe QDs confirmed their inefficacy as visible-light activated QD therapeutics (FIG. 16E). Despite its favourable CB position, ZnTe could not serve as a nanotherapeutic alternative to CdTe-2.4 because of its large bandgap in the ultraviolet (UV). UV is more harmful than visible light and less able to penetrate surfaces such as skin. In order to reduce the risk of cadmium toxicity while maintaining visible light photochemical properties, different Zn₁₋ₓCdₓTe QDs were synthesized (varying x, FIG. 16F), via a modified hydrothermal method. Using inductively coupled plasma mass spectrometry (ICP-MS), it was found that increased zinc loading in precursor solutions yields greater zinc incorporation (FIG. 17A). Precursor Zn²⁺:Cd²⁺ ratios used (1:1, 5:1, 10:1, 20:1) produced Zn₁₋ₓCdₓTe QDs of 1-x=0.04, 0.11, 0.63, and 0.68, respectively. High molar ratios of Zn²⁺ to Cd²⁺ are required because cadmium's reactivity with tellurium is higher than that of zinc. Zinc ultimately becomes the primary cation in the 10:1 and 20:1 mixtures, where the degree of zinc incorporation levels out near 70%. Precursor cation ratios above 20:1 did not produce stable QDs, likely due to a combination of high cation loading and ZnTe instability. In characterizing the different Zn₁₋ₓCdₓTe QDs, it was observed that zinc incorporation leads to electronic properties approaching those of ZnTe. Photoluminescence (PL) measurements show a clear blue-shift in PL peak position with increasing zinc content, as well as a concomitant decrease in fluorescence intensity (FIG. 17B). The same trend of blue-shifting is apparent from the UV-vis absorbance spectra (FIG. 22). These observations can be explained by the formation of ZnTe bonds in place of CdTe bonds. Compared to CdTe-2.4, ZnTe has shorter, stronger bonds. The greater covalent nature raises dislocation energies, thus shifting absorption and PL values toward higher energy blue wavelengths. Further, decreased fluorescence intensity likely results from intrinsic zinc defects or TeO facets on the QD surface.

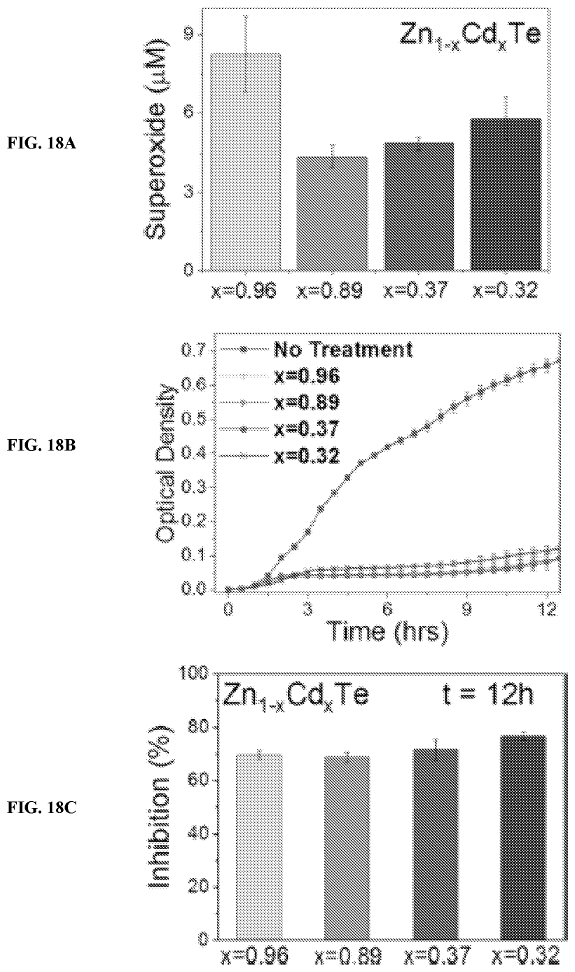

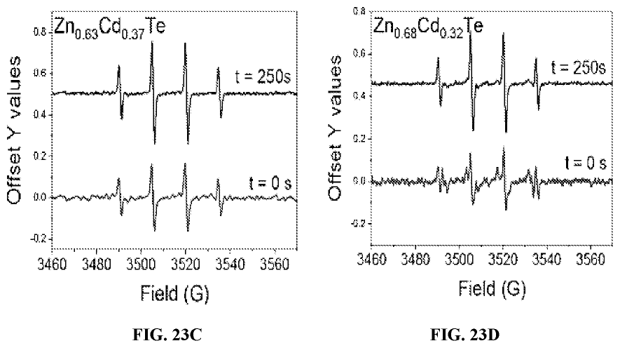

Following investigations of their bandgaps and optical properties, the electrochemical redox properties of composition-tuned Zn₁₋ₓCdₓTe QDs were characterized. As with the change from CdTe-2.4 to ZnTe QDs (x=1-0), it was expected that the CB would shift upward (higher energy) with increasing zinc content. Additionally, increasing x (cadmium element presence) should introduce lower energy states and impart a lower bandgap than that of ZnTe. Using DPV, it was confirmed that zinc incorporation increases the QD bandgap relative to CdTe by moving the CB (FIG. 17C). As predicted, all of the Zn₁₋ₓCdₓTe QDs have the electrochemical capacity for superoxide generation and have bandgaps significantly lower than that of ZnTe. EPR spectroscopy was used to verify radical generation upon excitation with visible light. After 45 s of illumination, EPR spectra show characteristic peaks corresponding to superoxide radical adducts (FIGS. 23A-23D). All samples, spanning different degrees of zinc incorporation, demonstrated a response to visible light (FIG. 18A). Most Zn₁₋ₓCdₓTe QDs with varying compositions showed comparable or higher superoxide generation than the CdTe-2.4 QDs (x=1) used as a positive control.

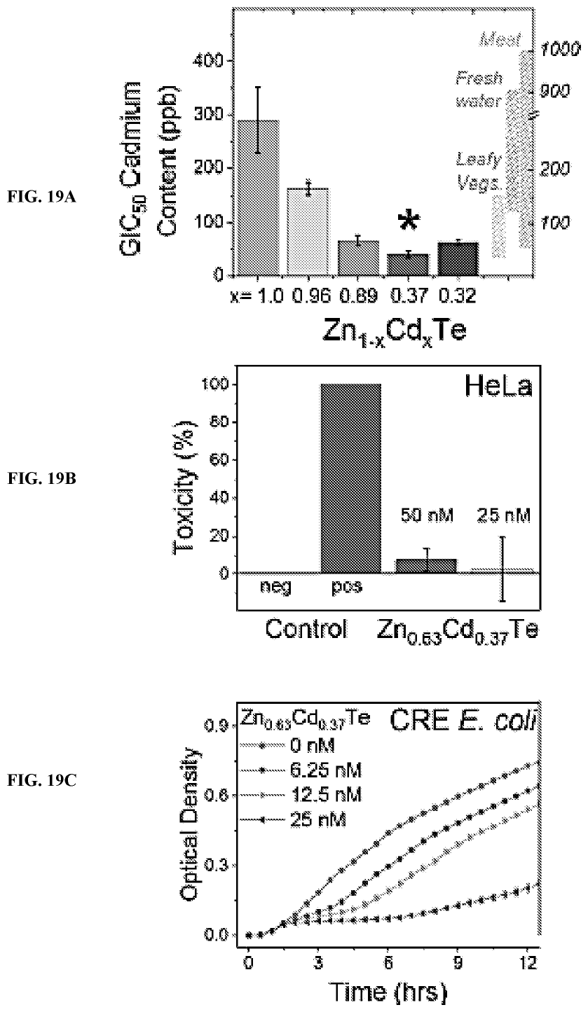

Intracellular superoxide generation is the mechanism of action and the strongest indicator of QD therapeutic effectiveness. Therefore, following the successful characterization of the visible bandgap, electronic states, electrochemical potential, and superoxide generation, the Zn₁₋ₓCdₓTe QD therapeutic effect was assessed in vitro. A cell culture of E. coli MG1655 treated with Zn₁₋ₓCdₓTe QDs in the presence of light was performed. In all four cases (1-x=0.04, 0.11, 0.63, and 0.68) Zn₁₋ₓCdₓTe QDs significantly inhibited bacterial growth (FIGS. 18B and 18C). Remarkably, these composition-tuned QDs retained the phototherapeutic effect of CdTe-2.4 with drastically lower levels of cadmium. To evaluate the extent of the cadmium content reduction while maintaining the same therapeutic effect, data was analyzed to develop a metric for the 50% growth inhibition concentration (GIC₅₀) multiplied by the cadmium content (in ppb via ICP-MS) for this therapeutic (FIG. 24). This provided a metric for cadmium content in the dose required for eliminating pathogenic bacteria, thereby maintaining effectiveness (super-oxide dosage) while minimizing cadmium content (FIG. 19A). Using CdTe-2.4 QDs as a positive control, modest effectiveness of the therapeutic led to low GIC₅₀ values, but the cadmium content was higher and would require ˜300 ppb of cadmium for a therapeutic effect. Decreasing the cadmium content while maintaining high effectiveness led to a rapid improvement; the cadmium required for the therapy reduced to lower than 50 ppb for Zn_{0.63}Cd_{0.37}Te QD therapy. However, further reducing cadmium content resulted in a gradual decrease in effectiveness and a resulting increase in GIC₅₀ values, thereby increasing the effective cadmium content for the same therapeutic action with Zn_{0.63}Cd_{0.37}Te QDs. Zn_{0.63}Cd_{0.37}Te QDs showed optimal therapeutic effect with the lowest cadmium content for effective therapy using visible light. Further elevated levels of zinc incorporation (ZnTe QDs) led to poor photoactivation with visible light and ineffective therapy.

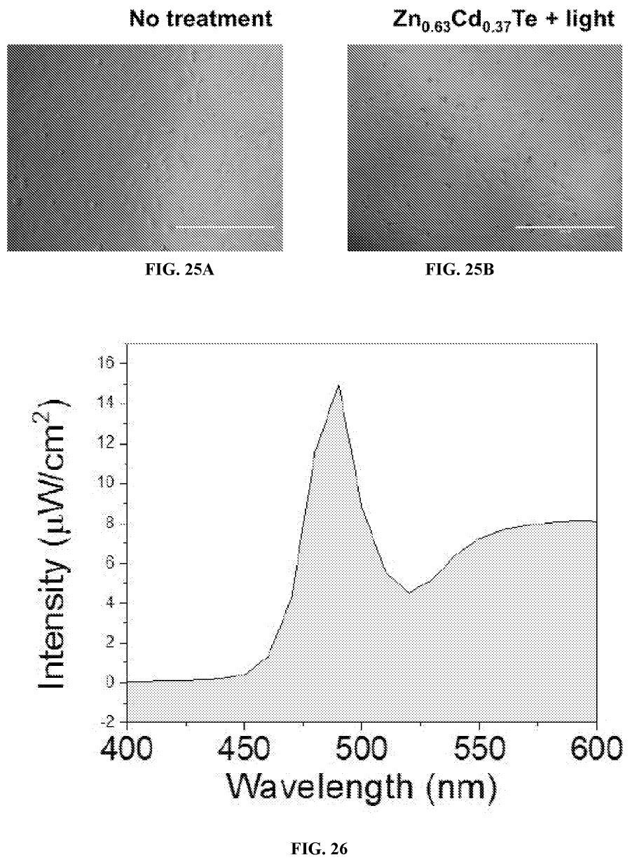

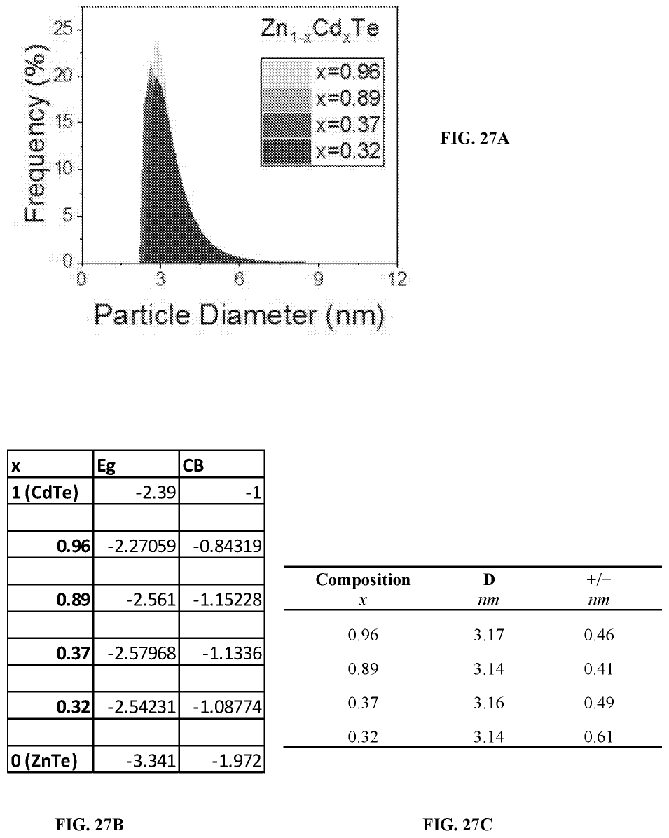

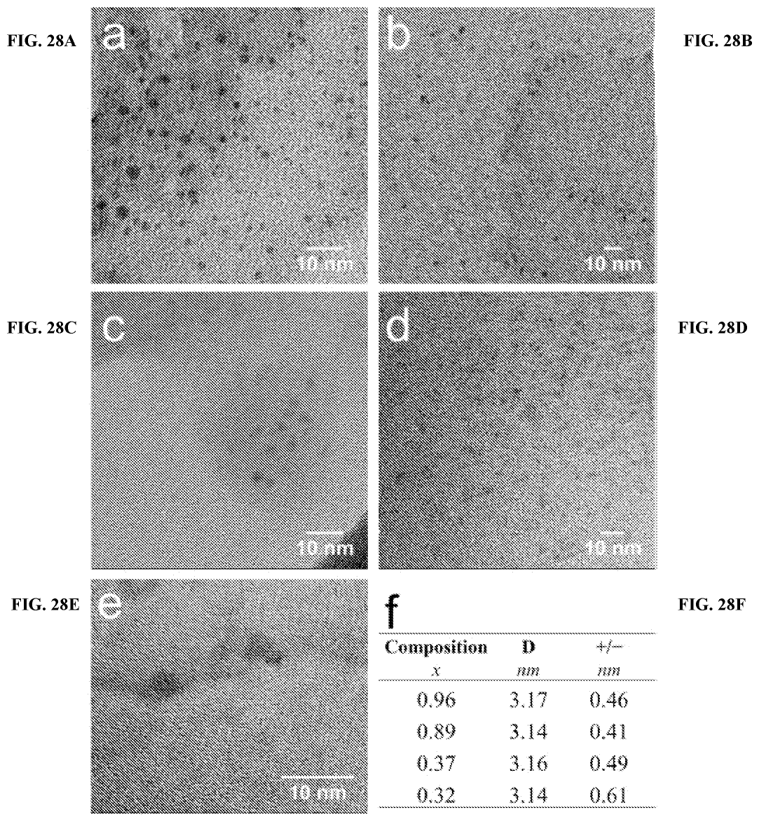

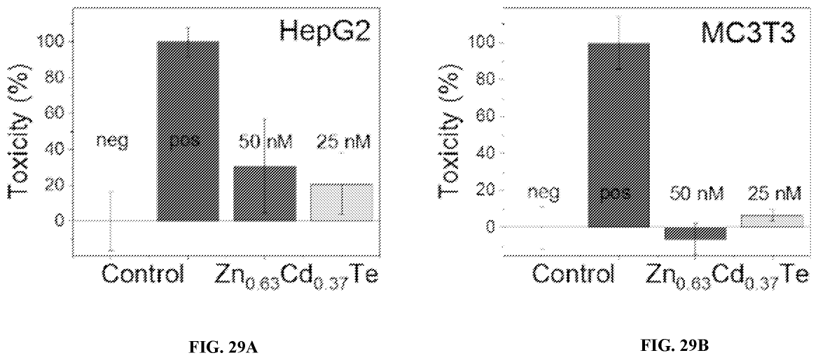

The observed phototherapeutic effect was directly attributable to superoxide generation in visible light. Superoxide, being a radical anion, is known to seek out enzymatic and free iron through electrostatic interactions and hence better able to target potential pathogens. Its stability and mechanism of action results in a targeted pathogen killing, unlike other ROS which cause nonspecific oxidative stress. Normally, intracellular superoxide concentrations are tightly controlled by the enzymesuperoxidedismutase(SOD). But this homeostasis is readily perturbed by exogenous sources of the radical; steady-state concentrations of 0.2 nM are known to inhibit bacterial growth. In this case, exogenous superoxide radicals formed by Zn₁₋ₓCdₓTe QDs overwhelmed the E. coli defences. Host biocompatibility was also an important metric in assessing these QDs. It was expected that Zn_{0.63}Cd_{0.37}Te would be biocompatible by considering the factors involved in nanoparticle-mediated toxicity: material toxicity, surface effects, and endogenous defense versus the mechanism of action. Replacing the majority of cadmium content by incorporating nontoxic zinc also improves innate host biocompatibility. The QDs described here are small and negatively charged (FIGS. 27 and 28, and Table 2), reducing the likelihood of unfavorable interactions from positively charged particles. Additionally, human cellular defense against superoxide is more robust at low concentrations, when compared to bacteria. Further, the cadmium content required for effective therapeutic dosage here contains less cadmium than other environmental factors like tubular potatoes and leafy vegetables, animal meat, or even fresh water (FIG. 19A). This suggested that a dosage deemed bactericidal may be reasonably nontoxic to human cell lines. Host biocompatibility using mammalian cell culture in the presence of photoactivated Zn₁₋ₓCdₓTe was evaluated. HeLa cells exposed to high doses of illuminated QDs were cultured in comparison to positive and negative toxicity controls. Using a lactate dehydrogenase (LDH) toxicity assay, it was found that Zn_{0.63}Cd_{0.37}Te QDs at therapeutic dosages (25 and 50 nM) were not toxic after 18 h of exposure (FIG. 19B). LDH detection for even the high dose of 50 nM was negligible compared to the no treatment condition. HeLa cell health was also evaluated by microscopy (FIGS. 25A-25B). As with the no treatment (negative control), HeLa cells exposed to photo-activated Zn_{0.63}Cd_{0.37}Te were still morphologically healthy. Such a “window of dosage” is the result of increased bacterial susceptibility to superoxide. Toxicity assays were repeated using two other human cell lines: osteoblasts (MC3T3-E1; bone-forming cells) and hepatic cells (HepG2-C3A; liver cells). Results of each assay were consistent with those observed using the HeLa cell line (FIGS. 29A-B); no dosage of Zn_{0.63}Cd_{0.37}Te QDs caused statistically significant toxicity to either human cell line. These results signalled a degree of effectiveness that can be used to fight multidrug resistant infections in humans.

TABLE 2
Measurement of colloidal stability (zeta potential)
and diameter (via dynamic light scattering) for
ternary quantum dots of varying composition.

| Zn₁₋ₓCdₓTe | Zeta | Diameter |
|---|---|---|
| x = 0.96 | −71.5 | 3.15 ± 0.25 |
| x = 0.89 | −57.45 | 3.01 ± 0.39 |
| x = 0.37 | −58.99 | 3.03 ± 0.46 |
| x = 0.32 | −68.9 | 3.3 ± 0.52 |

In order to assess the effectiveness of Zn₁₋ₓCdₓTe QDs in countering other MDR bacterial pathogens, a cell culture with a carbapenem-resistant E. coli clinical isolate was investigated. CRE E. coli is a “priority 1” critical pathogen, as designated by the World Health Organization. Upon treatment with Zn_{0.63}Cd_{0.37}Te, considerable growth inhibition at nanomolar doses (FIG. 19C) was observed. After 9 h of growth, Zn_{0.63}Cd_{0.37}Te QD treatment resulted in strong photo-inhibition (FIG. 19C). Photoinhibition was apparent, even at 6.25 nM, and it became more apparent as the dosage increased. A 25 nM dose of Zn_{0.63}Cd_{0.37}Te resulted in approximately 80% inhibition of the priority 1 antibiotic-resistant pathogen.

In some embodiments, x is 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, or 0.98.

In summary, it is demonstrated herein that the composition-tuning of ternary Zn₁₋ₓCdₓTe QDs and elucidated its effect on optical bandgap, electronic states, electrochemical potentials, generation of selective superoxide anions as a therapeutic, and the QD effectiveness in selectively killing “priority 1” MDR pathogen using visible light. These rationally designed Zn₁₋ₓCdₓTe QDs showed negligible toxicity in mammalian cells and generated intracellular superoxide radical using visible light to provide selective and effective therapy. To further quantify their effectiveness with reducing cadmium content, using CdTe-2.4 QDs as a positive control, our results indicate that, together with a nanomolar dosage, Zn₁₋ₓCdₓTe QDs provide considerable therapeutic effects with minimal cadmium content (GIC₅₀ (nM)×ppb/nM of Cd<50 ppb, close to the detection limit of our instrument). This work demonstrates the potential to design a nanotherapeutic that is more benign in its constituent materials and maintains high selectivity toward eliminating a broad range of MDR pathogens while demonstrating negligible effect on the growth, health, and metabolism of host mammalian cells. These results highlight the rational design approach toward developing other effective QD candidates for superoxide-mediated therapeutics to counter the growing threat of antimicrobial resistance and MDR bacterial infections.

### Enumerated Embodiments

The following exemplary embodiments are provided, the numbering of which is not to be construed as designating levels of importance.

Embodiment 1 provides a composition comprising at least one

a. indium phosphide (InP) quantum dot or

b. ternary zinc cadmium telluride (Zn₁₋ₓCdₓTe) quantum dot,

wherein the InP quantum dot or the Zn₁₋ₓCdₓTe quantum dot has a conduction band position above about −0.33 eV, as referenced to normal hydrogen electrode (NHE), at pH 7;

wherein the InP quantum dot or the Zn₁₋ₓCdₓTe quantum dot has a valence band position below about +1.0 eV, as referenced to NHE at pH 7; and

wherein is x a number greater than 0 and less than 1.

Embodiment 2 provides the composition of Embodiment 1, wherein when the diameter of the InP quantum dot is about 4 nm to about 7 nm, the InP quantum dot absorbs at wavelengths ranging from about 650 nm to about 700 nm.
Embodiment 3 provides the composition of Embodiments 1-2, wherein when the diameter of the InP quantum dot is about 5 nm to about 7 nm, the InP quantum dot absorbs at wavelengths ranging from about 700 nm to about 1000 nm.
Embodiment 4 provides the composition of Embodiments 1-3, wherein the conduction band position of the InP quantum dot is about −0.9 eV to about −1.2 eV, as referenced to NHE at pH 7.
Embodiment 5 provides the composition of Embodiments 1-4, wherein the conduction band position of the InP quantum dot is about −0.5 eV to about −0.8 eV, as referenced to NHE at pH 7.
Embodiment 6 provides the composition of Embodiments 1-5, wherein the InP quantum dot is at least partially coated with one selected from the group consisting of ZnS and ZnSe.
Embodiment 7 provides the composition of Embodiments 1-6, wherein the InP quantum dot has a band gap of about 0.9 eV to about 1.9 eV.
Embodiment 8 provides the composition of Embodiments 1-7, wherein x is a value selected from the group consisting of about 0.96, 0.89, 0.37, and about 0.32.
Embodiment 9 provides the composition of Embodiments 1-8, wherein the diameter of the Zn₁₋ₓCdₓTe quantum dot is about 2.9 nm to about 3.9 nm.
Embodiment 10 provides the composition of Embodiments 1-9, wherein the conduction band position of the Zn₁₋ₓCdₓTe quantum dot is about 1.0 to about 1.1 eV, as referenced to NHE at pH 7.
Embodiment 11 provides the composition of Embodiments 1-10, wherein the Zn₁₋ₓCdₓTe quantum dot has a band gap of about 2.3 eV to about 3.3 eV.
Embodiment 12 provides the composition of Embodiments 1-11, wherein the concentration of heavy metal in the composition comprising the InP quantum dot is less than about 0.0001% to about 1%.
Embodiment 13 provides the composition of Embodiments 1-12, wherein the quantum dots are at least partially coated with 3-mercaptopropionic acid.
Embodiment 14 provides a method of killing, preventing, or hampering the growth of a first cell, wherein the method comprises:
irradiating the composition of Embodiment 1 with electromagnetic radiation in the presence of a first cell, wherein the irradiation generates a therapeutically effective amount of superoxide radicals that kill the first cell and/or prevents or hampers the growth of the first cell.
   Embodiment 15 provides the method of Embodiment 14, wherein the composition comprises the InP quantum dot, and the wavelength of the electromagnetic radiation ranges from about 650 nm to about 1000 nm.
   Embodiment 16 provides the method of Embodiments 14-15, wherein the composition comprises the Zn₁₋ₓCdₓTe quantum dot and the wavelength of the electromagnetic radiation ranges from about 400 nm to about 700 nm.
   Embodiment 17 provides the method of Embodiments 14-16, wherein irradiating does not generate any other form of reactive oxygen species (ROS).
   Embodiment 18 provides the method of Embodiments 14-17, wherein the at least one first cell is a bacterium.
   Embodiment 19 provides the method of Embodiments 14-18, wherein the bacterium comprises at least one selected from the group consisting of Mycobacterium tuberculosis, K. pneumonia, E. coli, S. aureus, P. aeruginosa, A. baumannii, and S. typhimurium.
   Embodiment 20 provides the method of Embodiments 14-19, wherein the first cell comprises a Gram-negative bacterium, and wherein the first cell is further contacted with at least one Gram-negative antibacterial agent.
   Embodiment 21 provides the method of Embodiments 14-20, wherein the concentration of the antibacterial agent that is required to kill, or prevent or hamper the growth of, the first cell in presence of the at least one quantum dot is lower than the concentration of the antibacterial agent that is required to kill, or prevent or hamper the growth of the first cell when the antibacterial agent is used in the absence of the at least one quantum dot.
   Embodiment 22 provides the method of Embodiments 14-21, wherein the first cell is in the presence of at least one second cell, and wherein irradiation of the composition has no measurable effect on the growth, metabolism, or survival of the at least one second cell.
   Embodiment 23 provides the method of Embodiments 14-22, wherein the second cell is a mammalian cell.
   Embodiment 24 provides the method of Embodiments 14-23, wherein the second cell is a human cell.
   Embodiment 25 provides a method of killing bacteria, and/or preventing or hampering bacterial growth, in a subject in need thereof, wherein the method comprises:

administering to the subject a therapeutically effective amount of the composition of Embodiment 1; and

irradiating the quantum dot to generate a therapeutically effective amount of superoxide radical that kills, and/or prevents growth, and/or hampers growth of the bacteria.

Embodiment 26 provides the method of Embodiment 25, wherein the bacterium comprises at least one species selected from the group consisting of Mycobacterium tuberculosis, K. pneumonia, E. coli, S. aureus, P. aeruginosa, A. baumannii, and S. typhimurium.
Embodiment 27 provides the method of Embodiments 25-26, wherein the subject is a mammal.
Embodiment 28 provides the method of Embodiments 25-27, wherein the subject is a human.

### OTHER EMBODIMENTS

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this disclosure has been disclosed with reference to specific embodiments, it is apparent that other embodiments and variations of this disclosure may be devised by others skilled in the art without departing from the true spirit and scope of the disclosure. The appended claims are intended to be construed to include all such embodiments and equivalent variations.

What is claimed is:

1. A composition comprising at least one
a. indium phosphide (InP) quantum dot or
b. a ternary zinc cadmium telluride (Zn₁₋ₓCdₓTe) quantum dot,
wherein the InP quantum dot or the Zn₁₋ₓCdₓTe quantum dot has a conduction band position above about −0.33 eV, as referenced to normal hydrogen electrode (NHE), at pH 7;
wherein the InP quantum dot or the Zn₁₋ₓCdₓTe quantum dot has a valence band position below about +1.0 eV, as referenced to NHE at pH 7; and
wherein is x a number greater than 0 and less than 1.

2. The composition of claim 1, wherein at least one of the following is true:
i) the diameter of the InP quantum dot is about 4 nm to about 7 nm, and the InP quantum dot absorbs light at wavelengths ranging from about 650 nm to about 700 nm; or
ii) the diameter of the InP quantum dot is about 5 nm to about 7 nm, the InP quantum dot absorbs light at wavelengths ranging from about 700 nm to about 1000 nm;
iii) the conduction band position of the InP quantum dot is about −0.9 eV to about −1.2 eV, as referenced to NHE at pH 7;
iv) the conduction band position of the InP quantum dot is about −0.5 eV to about −0.8 eV, as referenced to NHE at pH 7.

3. (canceled)

4. (canceled)

5. (canceled)

6. The composition of claim 1, wherein the InP quantum dot is at least partially coated with one selected from the group consisting of ZnS and ZnSe.

7. The composition of claim 1, wherein the InP quantum dot has a band gap of about 0.9 eV to about 1.9 eV.

8. The composition of claim 1, wherein x is a value selected from the group consisting of about 0.96, 0.89, 0.37, and about 0.32.

9. The composition of claim 1, wherein the diameter of the Zn₁₋ₓCdₓTe quantum dot is about 2.9 nm to about 3.9 nm.

10. The composition of claim 9, wherein the conduction band position of the Zn₁₋ₓCdₓTe quantum dot is about 1.0 to about 1.1 eV, as referenced to NHE at pH 7.

11. The composition of claim 9, wherein the Zn₁₋ₓCdₓTe quantum dot has a band gap of about 2.3 eV to about 3.3 eV.

12. The composition of claim 1, wherein the concentration of heavy metal in the composition comprising the InP quantum dot is less than about 0.0001% to about 1%.

13. The composition of claim 1, wherein the quantum dots are at least partially coated with 3-mercaptopropionic acid.

14. A method of killing, preventing, or hampering the growth of a first cell, the method comprising:
irradiating the composition of claim 1 with electromagnetic radiation in the presence of a first cell, and
generating a therapeutically effective amount of superoxide radicals that kill the first cell or prevents or hamper the growth of the first cell.

15. The method of claim 14, wherein one of the following is true:
i) the composition comprises the InP quantum dot, and the wavelength of the electromagnetic radiation ranges from about 650 nm to about 1000 nm; or
ii) the composition comprises the Zn₁₋ₓCdₓTe quantum dot and the wavelength of the electromagnetic radiation ranges from about 400 nm to about 700 nm.

16. (canceled)

17. The method of claim 14, wherein irradiating does not generate any other form of reactive oxygen species (ROS).

18. The method of claim 14, wherein the at least one first cell is a bacterium.

19. The method of claim 18, wherein the bacterium comprises at least one selected from the group consisting of Mycobacterium tuberculosis, K. pneumonia, E. coli, S. aureus, P. aeruginosa, A. baumannii, and S. typhimurium.

20. The method of claim 18, wherein the first cell comprises a Gram-negative bacterium, and wherein the first cell is further contacted with at least one Gram-negative antibacterial agent.

21. The method of claim 20, wherein the concentration of the antibacterial agent that is required to kill, or prevent or hamper the growth of, the first cell in presence of the at least one quantum dot is lower than the concentration of the antibacterial agent that is required to kill, or prevent or hamper the growth of the first cell when the antibacterial agent is used in the absence of the at least one quantum dot.

22. The method of claim 14, wherein the first cell is in the presence of at least one mammalian cell, and wherein irradiation of the composition has no measurable effect on the growth, metabolism, or survival of the at least one mammalian cell.

23. (canceled)

24. (canceled)

25. A method of killing bacteria, or preventing or hampering bacterial growth in a mammalian subject in need thereof, the method comprising:
administering to the subject a therapeutically effective amount of the composition of claim 1; and
irradiating the quantum dot to generate a therapeutically effective amount of superoxide radical that kills, or prevents or hampers growth of the bacteria.

26. The method of claim 25, wherein the bacterium comprises at least one species selected from the group consisting of Mycobacterium tuberculosis, K. pneumonia, E. coli, S. aureus, P. aeruginosa, A. baumannii, and S. typhimurium.

27. (canceled)

28. (canceled)
